(19) United States
(12) Patent Application Publication
(10) Pub. No.: US 2022/0177878 A1
(43) Pub. Date: Jun. 9, 2022

(54) CRISPR/CAS9 GENE EDITING OF ATXN2 FOR THE TREATMENT OF SPINOCEREBELLAR ATAXIA TYPE 2

(71) Applicant: THE CHILDREN'S HOSPITAL OF PHILADELPHIA, Philadelphia, PA (US)

(72) Inventors: Bryan SIMPSON, Philadelphia, PA (US); Beverly DAVIDSON, Philadelphia, PA (US); Alejandro MAS MONTEYS, Philadelphia, PA (US)

(73) Assignee: THE CHILDREN'S HOSPITAL OF PHILADELPHIA, Philadelphia, PA (US)

(21) Appl. No.: 17/594,651

(22) PCT Filed: Apr. 24, 2020
(86) PCT No.: PCT/US2020/029804

§ 371 Date: Oct. 25, 2021

Related U.S. Application Data
(60) Provisional application No. 62/839,336, filed on Apr. 26, 2019.

(51) Int. Cl. C12N 15/11; C12N 15/113; C12N 15/90; C12N 15/86; C12N 9/22

(52) U.S. Cl. CPC C12N 15/111; C12N 15/113; C12N 15/90; C12N 15/86; C12N 2320/34; C12N 2310/20; C12N 2750/14143; C12N 2830/008; C12N 9/22

(57) ABSTRACT

CRISPR/Cas9-mediated genome editing holds clinical potential for treating genetic diseases, such as spinocerebellar ataxia type 2, which is caused by mutations in the ATXN2 gene. Here, compositions and methods for the treatment of spinocerebellar taxia type 2 are provided. In some embodiments, a composition providing a vector encoding a guide RNA of the disclosure and a vector encoding a Cas9 protein or nuclease domain thereof of the disclosure are provided for use in the treatment of spinocerebellar ataxia type 2.

### REFERENCE TO RELATED APPLICATIONS

The present application claims the priority benefit of U.S. provisional application No. 62/839,336, filed Apr. 26, 2019, the entire contents of which is incorporated herein by reference.

### STATEMENT REGARDING FEDERALLY SPONSORED RESEARCH

This invention was made with government support under grant no. R01 NS076631 awarded by the National Institutes of Health. The government has certain rights in the invention.

### REFERENCE TO A SEQUENCE LISTING

The instant application contains a Sequence Listing which has been submitted electronically in ASCII format and is hereby incorporated by reference in its entirety. Said ASCII copy, created on Apr. 24, 2020, is named “CHOPP0027WO_ST25.txt” and is 76.6 kilobytes in size.

### BACKGROUND

### 1. Field

The present disclosure relates to the fields of molecular biology, medicine, and genetics. More particularly, the disclosure relates to compositions and uses thereof for genome editing to correct mutations in vivo using an exon-skipping and/or reframing approach.

### 2. Related Art

Spinocerebellar ataxia type 2 (SCA2) is inherited in an autosomal dominant manner This means that having one changed (mutated) copy of A ATXN2 in each cell is enough to cause signs and symptoms of the condition. The ATXN2 gene mutations that cause SCA2 involve a DNA sequence called a ‘CAG trinucleotide repeat.’ It is made up of a series of three DNA building blocks (CAG stands for cytosine, adenine, and guanine) that appear multiple times in a row. The CAG sequence is normally repeated about 22 times in the gene, but it can be repeated up to 31 times without causing health problems. SCA2 develops in people who have 32 or more CAG repeats in the ATXN2 gene.

CRISPR sequences are Clustered Regularly Interspaced Short Palindromic Repeat sequences that are present in bacteria and archaea. CRISPR sequences work together with proteins from the Cas (CRISPR associated) group to form a kind of immune reaction against viral infections. Recently, it has been found that CRISPR sequences can also work together with a different enzyme, Cpf1. CRISPR is being studied intensely in the field of genetic disease where altering expression of target genes in vivo could potentially be therapeutic.

### SUMMARY

Despite intense efforts, there remains no cure for spinocerebellar ataxia type 2 (ATXN2). The disclosure provides compositions comprising an ATXN2 guide RNA-Cas9 complex for reducing the expression of the toxic ATXN2 mutant protein. Compositions and method of the disclosure may be used to treat spinocerebellar ataxia type 2.

In one embodiment, there is provided a composition comprising:
a first vector comprising a nucleic acid comprising:
   a sequence encoding at least a first guide RNA targeting a genomic target sequence according to SEQ ID NO: 1, 3, 4, 5, 6, 7 or 8;
   a sequence encoding a first promoter, wherein the first promoter drives expression of the sequence encoding the first guide RNA, and
a second vector comprising a nucleic acid comprising:
   a sequence encoding a Cas9 or a nuclease domain thereof;
   a sequence encoding a second promoter, wherein the second promoter drives expression of the sequence encoding a Cas9 or a nuclease domain thereof.
      The ratio of the first vector to the second vector in the composition may be between 1:1 and 1:30. The first vector may comprise at least one sequence encoding a second guide RNA targeting a genomic target sequence according to SEQ ID NO: 2, or SEQ ID NO: 1, 3, 4, 5, 6, 7 or 8 that differs from the first guide RNA; and at least one sequence encoding a third promoter, wherein the third promoter drives expression of the at least one sequence encoding the second guide RNA. The first vector and/or the second vector may comprise a sequence isolated or derived from an adeno-associated virus (AAV), such as adeno-associated virus 1 (AAV1).

The sequence encoding the first promoter may comprise or consist of a U6 promoter. The sequence encoding the third promoter may comprise or consist of a U6 promoter. The sequence encoding the second promoter may comprise or consist of a neuron-specific promoter, such as, for example, a Mecp2 promoter or a Syn1 promoter. The composition comprises between 5×10⁶ viral genomes (vg)/kilogram (kg) and 1×10¹⁵ vg/kg, inclusive of the endpoints, of the first vector. The composition may comprise 1×10⁷ viral genomes (vg)/kilogram (kg) to 1×10¹⁰ viral genomes (vg)/kilogram (kg), inclusive of the endpoints, of the first vector. The composition may further comprise a pharmaceutically acceptable carrier.

In another embodiment, there is provided a composition comprising a first vector comprising a nucleic acid comprising:
a sequence encoding at least a first guide RNA targeting a genomic target sequence according to SEQ ID NO: 1, 3, 4, 5, 6, 7 or 8;
a sequence encoding a first promoter, wherein the first promoter drives expression of the sequence encoding the first guide RNA.
   The first vector may comprise at least one sequence encoding a second guide RNA targeting a genomic target sequence according to SEQ ID NO: 2, or SEQ ID NO: 1, 3, 4, 5, 6, 7 or 8 that differs from the first guide RNA; and at least one sequence encoding a second promoter, wherein the second promoter drives expression of the at least one sequence encoding the second guide RNA. The first vector may comprise a sequence isolated or derived from an adeno-associated virus (AAV), such as adeno-associated virus 1 (AAV1).

The sequence encoding the first promoter may comprise or consist of a U6 promoter. The sequence encoding the second promoter may comprise or consist of a U6 promoter. The composition comprises between 5×10⁶ viral genomes (vg)/kilogram (kg) and 1×10¹⁵ vg/kg, inclusive of the endpoints, of the first vector. The composition may comprise 1×10⁷ viral genomes (vg)/kilogram (kg) to 1×10¹⁰ viral genomes (vg)/kilogram (kg), inclusive of the endpoints, of the first vector. The composition may further comprise a pharmaceutically acceptable carrier.

The first vector may further comprise a nucleic acid comprising:
a sequence encoding a Cas9 or a nuclease domain thereof;
a sequence encoding a third promoter, wherein the third promoter drives expression of the sequence encoding a Cas9 or a nuclease domain thereof.

In yet another embodiment, there is provided a method of preventing, treating, slowing the onset of, or slowing the progression of spinocerebellar ataxia type 2 in a subject in need thereof comprising administering to the subject a therapeutically effective amount a first vector comprising a nucleic acid comprising:
a sequence encoding at least a first guide RNA targeting a genomic target sequence according to SEQ ID NO: 1, 3, 4, 5, 6, 7 or 8;
a sequence encoding a first promoter, wherein the first promoter drives expression of the sequence encoding the first guide RNA, and
   a second vector comprising a nucleic acid comprising:
a sequence encoding a Cas9 or a nuclease domain thereof;
a sequence encoding a second promoter, wherein the second promoter drives expression of the sequence encoding a Cas9 or a nuclease domain thereof.
   The first vector may comprise at least one sequence encoding a second guide RNA targeting a genomic target sequence according to SEQ ID NO: 2, or SEQ ID NO: 1, 3, 4, 5, 6, 7 or 8 that differs from the first guide RNA; and at least one sequence encoding a third promoter, wherein the third promoter drives expression of the at least one sequence encoding the second guide RNA. The of the first vector to the second vector in the composition may be between 1:1 and 1:30.

The composition may be administered locally, such as to brain tissue, or systemically. The composition may be administered by an intravenous infusion or injection. The subject may be a neonate, an infant, a child, a young adult, or an adult. The subject may have been diagnosed as having spinocerebellar ataxia type 2 or may be a genetic carrier for spinocerebellar ataxia type 2. The subject may be male or female. The adult subject may be at least 18 years old, or at least 25 years old. The child subject may be less than 18 years of age, and/or may be 20 kg or less. The infant subject is an infant may be less than 2 years old.

The method may result, upon administering the therapeutically effective amount of the composition, in a minimal immune response to the composition in the subject. The effective amount may comprise between 5×10⁶ viral genomes (vg)/kilogram (kg) and 1×10¹⁵ vg/kg, inclusive of the endpoints, of the first vector. The composition may comprise 1×10⁷ viral genomes (vg)/kilogram (kg) to 1×10¹⁰ viral genomes (vg)/kilogram (kg), inclusive of the endpoints, of the first vector. Administration of the therapeutically effective amount of the composition may be provided as a single dose or provided within a single medical procedure. Administration of the therapeutically effective amount of the composition may be provided as multiple doses or provided over multiple medical procedures. The administration may result in the reduction of ATXN2 protein expression, but not ATXN2 mRNA, such as a reduction of ATXN2 protein expression by about 55%-70% as compared to pre-treatment levels in a targeted cell. The administration may result in the introduction of indels near an ATXN2 target sequence. The administration may result in the deletion of a CAG repeat in an ATXN2 target sequence.

In some embodiments of the method of treating of the disclosure, the administration of the therapeutically effective amount of the composition is provided as a single dose or provided within a single medical procedure.

In some embodiments of the method of treating of the disclosure, the administration of the therapeutically effective amount of the composition is provided as multiple doses or provided over multiple medical procedures.

As used herein in the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of±10% of the stated value.

Throughout this application, nucleotide sequences are listed in the 5′ to 3′ direction, and amino acid sequences are listed in the N-terminal to C-terminal direction, unless indicated otherwise.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Diagram of ATXN2 isoform variant 1, isoform variant 2 and isoform variant 3.

FIG. 2. Diagram of gRNA locations targeting Cas9 to ATXN2 exon 1.

FIG. 3A. gRNAs alone or in combination reduced ATXN2 protein levels in HEK293 cells.

FIG. 3B. gRNAs alone or in combination reduced ATXN2 protein levels in HEK293 cells.

FIG. 3C. gRNAs alone reduced ATXN2 protein levels in HEK293 cells.

FIG. 3D. gRNAs in combination reduced ATXN2 protein levels in HEK293 cells.

FIG. 4. PCR amplified products of genomic DNA isolated after editing in HEK293 cells show editing and deletion of the CAG repeat with gRNA2+3.

FIG. 5. Sanger sequencing of PCR amplified products after editing in HEK293 cells detected indels with gRNA3 and deletion of the CAG repeat with gRNA2+3. The sequences shown in the figure, from top to bottom, are SEQ ID NOs: 28-35, 2, and 1.

FIG. 6A. gRNAs alone or in combination reduced all ATXN2 isoforms in HEK293 cells.

FIG. 6B. gRNAs alone or in combination reduced ATXN2 isoform 2 in HEK293 cells.

FIG. 6C. gRNAs alone or in combination reduced ATXN2 isoform 3 in HEK293 cells.

FIG. 6D. gRNAs alone or in combination reduced ATXN2 isoform 1 in HEK293 cells.

FIG. 7. PCR amplified products of genomic DNA isolated after editing in pre-onset and post-onset BAC-ATXN2-Q72 mice shows deletion of the CAG repeat with gRNA2+3.

FIG. 8. Sanger sequencing of PCR amplified products after editing in pre-onset and post-onset BAC-ATXN2-Q72 mice detected deletion of the CAG repeat with gRNA2+3. The sequences in the figure for gRNA2+3 edited DNA and excised CAG repeat are, from top to bottom, SEQ ID NOs: 32, 33, 36, and 37, respectively. The sequence in the figure for gRNA 2 is SEQ ID NO: 2. The sequence in the figure for gRNA 3 is SEQ ID NO: 1. The sequences in the figure for mouse #233, #206, and #226 are SEQ ID NOs: 38-40, respectively.

FIG. 9. Dose-dependent reduction of hATXN2-Q72 levels after AAV1-Cas9 infusion into the cerebellum of pre-onset BAC-ATXN2-Q72 mice.

FIG. 10. Dose-dependent reduction of hATXN2-Q72 levels after AAV1-Cas9 infusion into the cerebellum of post-onset BAC-ATXN2-Q72 mice.

FIG. 11. Comparison between Mecp2 and Syn1 neuron-specific promoters driving SpCas9 expression similarly reduced hATXN2-Q72 levels after dual AAV1 co-infusion with gRNA3 in the cerebellum of BAC-SCA2 mice.

### DETAILED DESCRIPTION

The disclosure provides Clustered Regularly Interspaced Short Palindromic Repeat/Cas9 (CRISPR/Cas9)-mediated genome editing compositions for reducing expression of ATXN2, which left untreated results in spinocerebellar ataxia type 2. The data presented herein show that in vivo AAV-mediated delivery of gene-editing components successfully reduce ATXN2 expression. More specifically, SpCas9 employed with gRNA3 creates indels near the target site of ATXN2, while a SpCas9 used with gRNA2 and gRNA3 together deletes the CAG repeat. Moreover, SpCa9 reduced ATXN2 protein levels in vitro with both gRNA3 alone, and the combination of gRNA2 and gRNA3. Further screening of gRNAs identified gRNA1, gRNA4, gRNA5, gRNA6, gRNA7 and gRNA8 that reduced ATXN2 protein levels alone or in combination with two gRNAs. Additionally, the inventors screened these gRNAs for knockdown of ATXN2 mRNA levels, specifically ATXN2 isoform variant 1, which is known to contain the CAG repeat, or all ATXN2 isoforms, including ATXN2 isoform variant 2 and variant 3. In vivo AAV1 delivery results in widespread biodistribution in mice, deletion of the expanded CAG repeat, and resulted in dose-dependent gene editing that reduced mutant ATXN2-Q72 expression when delivered to the cerebellum of pre-onset and post-onset BAC-ATXN2-Q72 mice.

Compositions and methods for treating spinocerebellar ataxia type 2 are provided herein. In some embodiments, an AAV construct is provided, wherein the AAV construct comprises a nucleic acid encoding one or more promoters to drive expression of at least one ATXN2 guide RNA. These and other aspects of the disclosure are reproduced below.

### I. CRISPR SYSTEMS

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

### A. Guide RNA (gRNA)

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100bp at the minimum length and contain a portion which is targets the 20 protospacer nucleotides immediately preceding the PAM sequence NGG; gRNAs do not contain a PAM sequence.

In some embodiments, the gRNA is gRNA3 correspond to the target sequence GCCAATGTCCGCAAGCCCGG (SEQ ID NO: 1). In other embodiments, the gRNA is gRNA2 correspond to the target sequence CCGCCCTCCGATGCGCTCAG (SEQ ID NO: 2). In other embodiments, the gRNA is gRNA1 correspond to the target sequence GCGAGACTCGGTGGCCACCG (SEQ ID NO: 3). In other embodiments, the gRNA is gRNA4 correspond to the target sequence ACCAAAACAGTCTGAGGCGG (SEQ ID NO: 4). In other embodiments, the gRNA is gRNA5 correspond to the target sequence GTCGCCGCGACCACCGAGGA (SEQ ID NO: 5). In other embodiments, the gRNA is gRNA6 correspond to the target sequence GGTCGCCGCGACCACCGAGG (SEQ ID NO: 6). In other embodiments, the gRNA is gRNA7 correspond to the target sequence GTGACCCGCCGGGCTACCCG (SEQ ID NO: 7). In other embodiments, the gRNA is gRNA8 correspond to the target sequence GCCCACCCCGGGTAGCCCGG (SEQ ID NO: 8).

### B. Nucleases

### 1. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (E coli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. Jinek et al. (2012) combined tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Wang et al. (2013) showed that coinjection of Cas9 mRNA and sgRNAs into the germline (zygotes) generated mice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wildtype or full length Cas9. In some embodiments the Cas9 is a spCas9 (AddGene).

### 2. Cpf1 Nucleases

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

In embodiments, the Cpf1 is a Cpf1 enzyme from Acidaminococcus (species BV3L6, UniProt Accession No. U2UMQ6; SEQ ID NO: 9), having the sequence set forth below:

| | |
|---|---|
| 1 | MTQFEGFTNL YQVSKTLRFE LIPQGKTLKH IQEQGFIEED KARNDHYKEL KPIIDRIYKT |
| 61 | YADQCLQLVQ LDWENLSAAI DSYRKEKTEE TRNALIEEQA TYRNAIHDYF IGRIDNLIDA |
| 121 | INKRHAEIYK GLFKAELFNG KVLKQLGTVT TTEHENALLR SFDKFTTYFS GFYENRKNVF |
| 181 | SAEDISTAIP HRIVQDNFPK FKENCHIFTR LITAVPSLRE HFENVKKAIG IFVSTSIEEV |
| 241 | FSFPFYNQLL TQTQIDLYNQ LLGGISREAG TEKIKGLNEV LNLAIQKNDE TAHIIASLPH |
| 301 | RFIPLFKQIL SDRNTLSFIL EEFKSDEEVI QSFCKYKTLL RNENVLETAE ALFNELNSID |
| 361 | LTHIFISHKK LETISSALCD HWDTLRNALY ERRISELTGK ITKSAKEKVQ RSLKHEDINL |
| 421 | QEIISAAGKE LSEAFKQKTS EILSHAHAAL DQPLPTTLKK QEEKEILKSQ LDSLLGLYHL |
| 481 | LDWFAVDESN EVDPEFSARL TGIKLEMEPS LSFYNKARNY ATKKPYSVEK FKLNFQMPTL |
| 541 | ASGWDVNKEK NNGAILFVKN GLYYLGIMPK QKGRYKALSF EPTEKTSEGF DKMYYDYFPD |
| 601 | AAKMIPKCST QLKAVTAHFQ THTTPILLSN NFIEPLEITK EIYDLNNPEK EPKKFQTAYA |
| 661 | KKTGDQKGYR EALCKWIDFT RDFLSKYTKT TSIDLSSLRP SSQYKDLGEY YAELNPLLYH |
| 721 | ISFQRIAEKE IMDAVETGKL YLFQIYNKDF AKGHHGKPNL HTLYWTGLFS PENLAKTSIK |
| 781 | LNGQAELFYR PKSRMKRMAH RLGEKMLNKK LKDQKTPIPD TLYQELYDYV NHRLSHDLSD |
| 841 | EARALLPNVI TKEVSHEIIK DRRFTSDKFF FHVPITLNYQ AANSPSKFNQ RVNAYLKEHP |
| 901 | ETPIIGIDRG ERNLIYITVI DSTGKILEQR SLNTIQQFDY QKKLDNREKE RVAARQAWSV |
| 961 | VGTIKDLKQG YLSQVIHEIV DLMIHYQAVV VLENLNFGFK SKRTGIAEKA VYQQFEKMLI |
| 1021 | DKLNCLVLKD YPAEKVGGVL NPYQLTDQFT SFAKMGTQSG FLFYVPAPYT SKIDPLTGFV |
| 1081 | DPFVWKTIKN HESRKHFLEG FDFLHYDVKT GDFILHFKMN RNLSFQRGLP GFMPAWDIVF |
| 1141 | EKNETQFDAK GTPFIAGKRI VPVIENHRFT GRYRDLYPAN ELIALLEEKG IVFRDGSNIL |
| 1201 | PKLLENDDSH AIDTMVALIR SVLQMRNSNA ATGEDYINSP VRDLNGVCFD SRFQNPEWPM |
| 1261 | DADANGAYHI ALKGQLLLNH LKESKDLKLQ NGISNQDWLA YIQELRN. |

In some embodiments, the Cpf1 is a Cpf1 enzyme from Lachnospiraceae (species ND2006, UniProt Accession No. A0A182DWE3; SEQ ID NO: 10), having the sequence set forth below:

| | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSFNGFTT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK. |

In some embodiments, the Cpf1 is codon optimized for expression in mammalian cells. In some embodiments, the Cpf1 is codon optimized for expression in human cells or mouse cells.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have an HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 does not require a tracrRNA, therefore, only crRNA is required. This benefits genome editing because Cpf1 is not only smaller than Cas9, but also it has a smaller sgRNA molecule (proximately half as many nucleotides as Cas9).

The Cpf1-crRNA complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system consist of a Cpf1 enzyme and a guide RNA that finds and positions the complex at the correct spot on the double helix to cleave target DNA. CRISPR/Cpf1 systems activity has three stages: 1) Adaptation, during which Cas1 and Cas2 proteins facilitate the adaptation of small fragments of DNA into the CRISPR array; 2) Formation of crRNAs: processing of pre-cr-RNAs producing of mature crRNAs to guide the Cas protein; and 3) Interference, in which the Cpf1 is bound to a crRNA to form a binary complex to identify and cleave a target DNA sequence.

### 3. Cas9 Versus Cpf1

Cas9 requires two RNA molecules to cut DNA while Cpf1 needs one. The proteins also cut DNA at different places, offering researchers more options when selecting an editing site. Cas9 cuts both strands in a DNA molecule at the same position, leaving behind ‘blunt’ ends. Cpf1 leaves one strand longer than the other, creating ‘sticky’ ends that are easier to work with. Cpf1 appears to be more able to insert new sequences at the cut site, compared to Cas9. Although the CRISPR/Cas9 system can efficiently disable genes, it is challenging to insert genes or generate a knock-in. Cpf1 lacks tracrRNA, utilizes a T-rich PAM and cleaves DNA via a staggered DNA DSB.

In summary, important differences between Cpf1 and Cas9 systems are that Cpf1 recognizes different PAMs, enabling new targeting possibilities, creates 4-5 nt long sticky ends, instead of blunt ends produced by Cas9, enhancing the efficiency of genetic insertions and specificity during NHEJ or HDR, and cuts target DNA further away from PAM, further away from the Cas9 cutting site, enabling new possibilities for cleaving the DNA.

TABLE 1
Differences between Cas9 and Cpf1

| Feature | Cas9 | Cpf1 |
|---|---|---|
| Structure | Two RNA required (Or 1 fusion | One RNA |
| | transcript (crRNA + tracrRNA = | required |
| | gRNA)) | |
| Cutting | Blunt end cuts | Staggered |
| mechanism | | end cuts |
| Cutting site | Proximal to recognition site | Distal from |
| | | recognition |
| | | site |
| Target sites | G-rich PAM | T-rich PAM |

### C. CRISPR-Mediated Gene Editing

The first step in editing the ATXN2 gene using CRISPR/Cpf1 or CRISPR/Cas9 is to identify the genomic target sequence. The genomic target for the gRNAs of the disclosure can be a ˜20 nucleotide DNA sequence, provided that the sequence is unique compared to the rest of the genome. The gRNAs of the application are described above.

The next step in editing the ATXN2 gene is to identify all Protospacer Adjacent Motif (PAM) sequences within the genetic region to be targeted. The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g., CRISPRdirect, available at world-wide-web at crispr.dbcls.jp).

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen. The gRNAs for Cpf1 are notably simpler than the gRNAs for Cas9, and only consist of a single crRNA containing direct repeat scaffold sequence followed by ˜20 nucleotides of guide sequence.

Each gRNA should then be validated in one or more target cell lines. For example, after the Cas9 or Cpf1 and the gRNA are delivered to the cell, the genomic target region may be amplified using PCR and sequenced according to methods known to those of skill in the art.

In some embodiments, gene editing may be performed in vitro or ex vivo. In some embodiments, the cells are contacted with one or more nucleic acids encoding the Cas9 or Cpf1and the guide RNA. In some embodiments, the one or more nucleic acids are introduced into the cells using, for example, lipofection or electroporation. Gene editing may also be performed in zygotes. In embodiments, zygotes may be injected with one or more nucleic acids encoding Cas9 or Cpf1 and a gRNA that targets the ATXN2 gene. The zygotes may subsequently be injected into a host.

In some embodiments, the Cas9 or Cpf1 is provided on a vector. In embodiments, the vector contains a Cas9 derived from S. pyogenes (SpCas9). In embodiments, the vector contains a Cas9 derived from S. aureus (SaCas9). In embodiments, the vector contains a Cpf1 sequence derived from a Lachnospiraceae bacterium. See, for example, Uniprot Accession No. A0A182DWE3. In embodiments, the vector contains a Cpf1 sequence derived from an Acidaminococcus bacterium. See, for example, Uniprot Accession No. U2UMQ6. In some embodiments, the Cas9 or Cpf1 sequence is codon optimized for expression in human cells or mouse cells. In some embodiments, the vector further contains a sequence encoding a fluorescent protein, such as GFP, which allows Cas 9 or Cpf1-expressing cells to be sorted using fluorescence activated cell sorting (FACS). In some embodiments, the vector is a viral vector such as an adeno-associated viral vector.

In some embodiments, the gRNA is provided on a vector. In some embodiments, the vector is a viral vector such as an adeno-associated viral vector. In embodiments, the Cas9 or Cpf1 and the guide RNA are provided on the same vector. In embodiments, the Cas9 or Cpf1 and the guide RNA are provided on different vectors.

Efficiency of in vitro or ex vivo Cas9 or Cpf1-mediated DNA cleavage may be assessed using techniques known to those of skill in the art, such as the T7 E1 assay. Reduction of ATXN2 expression may be confirmed using techniques known to those of skill in the art, such as RT-PCR, western blotting, and immunocytochemistry.

In some embodiments, contacting the cell with the Cas9 or the Cpf1 and the gRNA reduces ATXN2 expression. In embodiments, cells which have been edited in vitro or ex vivo, or cells derived therefrom, show levels of ATXN2 protein below pre-treated cells. In embodiments, the edited cells, or cells derived therefrom, express ATXN2 at a level that is 75%, 60%, 50%, 40%, 30%, 25%, 20%, 15%, 10%, 5% or 1% or any percentage in between of the pre-treatment ATXN2 expression levels.

### II. NUCLEIC ACID EXPRESSION VECTORS

As discussed above, in certain embodiments, expression cassettes are employed to express a transcription factor product, either for subsequent purification and delivery to a cell/subject, or for use directly in a genetic-based delivery approach. Provided herein are expression vectors which contain one or more nucleic acids encoding Cas9 or Cpf1 and at least one guide RNA that targets ATXN2. In some embodiments, a nucleic acid encoding Cas9 or Cpf1 and a nucleic acid encoding at least one guide RNA are provided on the same vector. In further embodiments, a nucleic acid encoding Cas9 or Cpf1 and a nucleic acid encoding least one guide RNA are provided on separate vectors.

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

### A. RNA Polymerase and Pol III Promoters

In eukaryotes, RNA polymerase III (also called Pol III) transcribes DNA to synthesize ribosomal 5S rRNA, tRNA and other small RNAs. The genes transcribed by RNA Pol III fall in the category of “housekeeping” genes whose expression is required in all cell types and most environmental conditions. Therefore, the regulation of Pol III transcription is primarily tied to the regulation of cell growth and the cell cycle, thus requiring fewer regulatory proteins than RNA polymerase II. Under stress conditions however, the protein Maf1 represses Pol III activity.

In the process of transcription (by any polymerase) there are three main stages: (i) initiation, requiring construction of the RNA polymerase complex on the gene's promoter; (ii) elongation, the synthesis of the RNA transcript; and (iii) termination, the finishing of RNA transcription and disassembly of the RNA polymerase complex.

Promoters under the control of RNA Pol III include those for ribosomal 5S rRNA, tRNA and few other small RNAs such as U6 spliceosomal RNA, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs.

### B. Additional Promoters and Elements

In some embodiments, the Cas9 or Cpf1 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promoters such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter

Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, a neuron-specific promoter (such as the Mecp2 promoter or the Syn1 promoter), immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone α gene. In some embodiments, the inducer is phorbol ester (TFA), heavy metals, glucocorticoids, poly(rI)x, poly(rc), E1A, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

### III. THERAPEUTIC COMPOSITIONS

### A. AAV-Cas9 Vectors

In some embodiments, a Cas9 may be packaged into an AAV vector. In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof.

Exemplary AAV-Cas9 vectors contain two ITR (inverted terminal repeat) sequences which flank a central sequence region comprising the Cas9 sequence. In some embodiments, the ITRs are isolated or derived from an AAV vector of serotype AAV1, AAV2, AAV3, AAV4, AAV5, AAV6, AAV7, AAV8, AAV9, AAV10, AAV11 AAVrh74, AAVrh10 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs.

In some embodiments, the AAV-Cas9 vector may contain one or more nuclear localization signals (NLS). In some embodiments, the AAV-Cas9 vector contains 1, 2, 3, 4, or 5 nuclear localization signals. Exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 11) of the IBB domain from importin-alpha, the sequences VSRKRPRP (SEQ ID NO: 12) and PPKKARED (SEQ ID NO: 13) of the myoma T protein, the sequence PQPKKKPL (SEQ ID NO: 14) of human p53, the sequence SALIKKKKKMAP (SEQ ID NO: 15) of mouse c-abl IV, the sequences DRLRR (SEQ ID NO: 16) and KQKKRK (SEQ ID NO: 17) of the influenza virus NS1, the sequence RKLKKKIKKL (SEQ ID NO: 18) of the Hepatitis virus delta antigen and the sequence REKKKFLKRR (SEQ ID NO: 19) of the mouse Mx1 protein. Further acceptable nuclear localization signals include bipartite nuclear localization sequences such as the sequence KRKGDEVDGVDEVAKKKSKK (SEQ ID NO: 20) of the human poly(ADP-ribose) polymerase or the sequence RKCLQAGMNLEARKTKK (SEQ ID NO: 21) of the steroid hormone receptors (human) glucocorticoid.

In some embodiments, the AAV-Cas9 vector may comprise additional elements to facilitate packaging of the vector and expression of the Cas9. In some embodiments, the AAV-Cas9 vector may comprise a polyA sequence. In some embodiments, the polyA sequence may be a mini-polyA sequence. In some embodiments, the AAV-Cas9 vector may comprise a transposable element. In some embodiments, the AAV-Cas9 vector may comprise a regulator element. In some embodiments, the regulator element is an activator or a repressor.

In some embodiments, the AAV-Cas9 may contain one or more promoters. In some embodiments, the one or more promoters drive expression of the Cas9.

In some embodiments, the AAV-Cas9 vector may be optimized for production in yeast, bacteria, insect cells, or mammalian cells. In some embodiments, the AAV-Cas9 vector may be optimized for expression in human cells. In some embodiments, the AAV-Cas9 vector may be optimized for expression in a bacculovirus expression system.

### B. AAV-sgRNA Vectors

In some embodiments, at least a first sequence encoding a gRNA and a second sequence encoding a gRNA may be packaged into an AAV vector. In some embodiments, at least a first sequence encoding a gRNA, a second sequence encoding a gRNA, and a third sequence encoding a gRNA may be packaged into an AAV vector. In some embodiments, at least a first sequence encoding a gRNA, a second sequence encoding a gRNA, a third sequence encoding a gRNA, and a fourth sequence encoding a gRNA may be packaged into an AAV vector. In some embodiments, at least a first sequence encoding a gRNA, a second sequence encoding a gRNA, a third sequence encoding a gRNA, a fourth sequence encoding a gRNA, and a fifth sequence encoding a gRNA may be packaged into an AAV vector. In some embodiments, a plurality of sequences encoding a gRNA are packaged into an AAV vector. For example, 1, 2, 3, 4, 5, 6, 7, 8, 9, 10, 11, 12, 13, 14, 15, 16, 17, 18, 19, or 20 sequences encoding a gRNA may be packaged into an AAV vector. In some embodiments, each sequence encoding a gRNA is different. In some embodiments, at least 1, 2, 3, 4, 5, 6, 7, 8, 9, 10, 11, 12, 13, 14, 15, 16, 17, 18, 19, or 20 of the sequences encoding a gRNA are the same. In some embodiments, all of the sequence encoding a gRNA are the same.

In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof.

Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof. In some embodiments, the ITRs are isolated or derived from an AAV vector of a first serotype and a sequence encoding a capsid protein of the AAV-sgRNA vector is isolated or derived from an AAV vector of a second serotype. In some embodiments, the first serotype and the second serotype are the same. In some embodiments, the first serotype and the second serotype are not the same. In some embodiments, the first serotype is AAV1, AAV2, AAV3, AAV4, AAV5, AAV6, AAV7, AAV8, AAV9, AAV10, AAV11, AAVrh74, or AAVrh10. In some embodiments, the second serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2 and the second serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2 and the second serotype is AAV9.

Exemplary AAV-sgRNA vectors contain two ITR (inverted terminal repeat) sequences which flank a central sequence region comprising the gRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof. In some embodiments, a first ITR is isolated or derived from an AAV vector of a first serotype, a second ITR is isolated or derived from an AAV vector of a second serotype and a sequence encoding a capsid protein of the AAV-sgRNA vector is isolated or derived from an AAV vector of a third serotype. In some embodiments, the first serotype and the second serotype are the same. In some embodiments, the first serotype and the second serotype are not the same. In some embodiments, the first serotype, the second serotype, and the third serotype are the same. In some embodiments, the first serotype, the second serotype, and the third serotype are not the same. In some embodiments, the first serotype is AAV1, AAV2, AAV3, AAV4, AAV5, AAV6, AAV7, AAV8, AAV9, AAV10, AAV11, AAVrh74, or AAVrh10. In some embodiments, the second serotype is AAV1, AAV2, AAV3, AAV4, AAV5, AAV6, AAV7, AAV8, AAV9, AAV10, AAV11, AAVrh74, or AAVrh10. In some embodiments, the third serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2, the second serotype is AAV4 and the third serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2, the second serotype is AAV4 and the third serotype is AAV9. Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAVrh74, or AAVrh10 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs.

In some embodiments, the AAV-sgRNA vector may comprise additional elements to facilitate packaging of the vector and expression of the sgRNA. In some embodiments, the AAV-sgRNA vector may comprise a transposable element. In some embodiments, the AAV-sgRNA vector may comprise a regulatory element. In some embodiments, the regulatory element comprises an activator or a repressor. In some embodiments, the AAV-sgRNA sequence may comprise a non-functional or “stuffer” sequence. Exemplary stuffer sequences of the disclosure may have some (a non-zero percentage of) identity or homology to a genomic sequence of a mammal (including a human). Alternatively, exemplary stuffer sequences of the disclosure may have no identify or homology to a genomic sequence of a mammal (including a human). Exemplary stuffer sequences of the disclosure may comprise or consist of naturally occurring non-coding sequences or sequences that are neither transcribed nor translated following administration of the AAV vector to a subject.

In some embodiments, the AAV-sgRNA vector may be optimized for production in yeast, bacteria, insect cells, or mammalian cells. In some embodiments, the AAV-sgRNA vector may be optimized for expression in human cells. In some embodiments, the AAV-Cas9 vector may be optimized for expression in a bacculovirus expression system.

In some embodiments, the AAV-sgRNA vector comprises at least one promoter. In some embodiments, the AAV-sgRNA vector comprises at least two promoters. In some embodiments, the AAV-sgRNA vector comprises at least three promoters. In some embodiments, the AAV-sgRNA vector comprises at least four promoters. In some embodiments, the AAV-sgRNA vector comprises at least five promoters. Exemplary promoters include, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), SV40, polyoma, retroviruses, papilloma virus, hepatitis B virus, human immunodeficiency virus, cytomegalovirus (CMV), and gibbon ape leukemia virus. Further exemplary promoters include the U6 promoter, the H1 promoter, and the 7SK promoter.

In some embodiments, the AAV vector comprises a first sequence encoding a gRNA and a second sequence encoding a gRNA, a first promoter drives expression of the first sequence encoding a gRNA and a second promoter drives expression of the second sequence encoding a gRNA. In some embodiments, the first and second promoters are the same. In some embodiments, the first and second promoters are different.

In some embodiments, the AAV vector comprises a first sequence encoding a gRNA, a second sequence encoding a gRNA, and a third sequence encoding a gRNA, a first promoter drives expression of the first sequence encoding a gRNA, a second promoter drives expression of the second sequence encoding a gRNA, and a third promoter drives expression of a third sequence encoding a gRNA. In some embodiments, at least two of the first, second, and third promoters are the same. In some embodiments, each of the first, second, and third promoters are different. In some embodiments, the first sequence encoding a gRNA, the second sequence encoding a gRNA, and the third sequence encoding a gRNA are identical. In some embodiments, the first sequence encoding a gRNA, the second sequence encoding a gRNA, and the third sequence encoding a gRNA are not identical.

In some embodiments, the AAV vector comprises a first sequence encoding a gRNA, a second sequence encoding a gRNA, a third sequence encoding a gRNA, and a fourth sequence encoding a gRNA, a first promoter drives expression of the first sequence encoding a gRNA, a second promoter drives expression of the second sequence encoding a gRNA, a third promoter drives expression of the third sequence encoding a gRNA, and a fourth promoter drives expression of the fourth sequence encoding a gRNA. In some embodiments, at least two of the first, second, third, and fourth promoters are the same. In some embodiments, each of the first, second, third, and fourth promoters are different. In some embodiments, the first sequence encoding a gRNA, the second sequence encoding a gRNA, the third sequence encoding a gRNA, and the fourth sequence encoding a gRNA are identical. In some embodiments, the first sequence encoding a gRNA, the second sequence encoding a gRNA, the third sequence encoding a gRNA, and the fourth sequence encoding a gRNA are not identical.

In some embodiments, the AAV vector comprises a first sequence encoding a gRNA, a second sequence encoding a gRNA, a third sequence encoding a gRNA, a fourth sequence encoding a gRNA, and a fifth sequence encoding a gRNA, a first promoter drives expression of the first sequence encoding a gRNA, a second promoter drives expression of the second sequence encoding a gRNA, a third promoter drives expression of the third sequence encoding a gRNA, a fourth promoter drives expression of the fourth sequence encoding a gRNA, and a fifth promoter drives expression of the fifth sequence encoding a gRNA. In some embodiments, at least two of the first, second, third, fourth, and fifth promoters are the same. In some embodiments, each of the first, second, third, fourth, and fifth promoters are different. In some embodiments, each of the first, second, third, and fourth promoters are different. In some embodiments, the first sequence encoding a gRNA, the second sequence encoding a gRNA, the third sequence encoding a gRNA, the fourth sequence encoding a gRNA, and the fifth sequence encoding a gRNA are identical. In some embodiments, the first sequence encoding a gRNA, the second sequence encoding a gRNA, the third sequence encoding a gRNA, the fourth sequence encoding a gRNA, and the fifth sequence encoding a gRNA are not identical.

### IV. PHARMACEUTICAL COMPOSITIONS AND DELIVERY METHODS

Also provided herein are compositions comprising one or more vectors and/or nucleic acids of the disclosure. In some embodiments, the composition further comprises a pharmaceutically acceptable carrier.

For clinical applications, pharmaceutical compositions are prepared in a form appropriate for the intended application. Generally, this entails preparing compositions that are essentially free of pyrogens, as well as other impurities that could be harmful to humans or animals

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, intracranial, intra-cerebellum, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine) and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, a first vector and a second vector are administered to a patient. In some embodiments, the first vector comprises a nucleic acid comprising a first sequence encoding a first guide RNA targeting a first genomic target sequence; a sequence encoding a second guide RNA targeting a second genomic target sequence; a sequence encoding a first promoter, wherein the first promoter drives expression of the sequence encoding the first guide RNA; and a sequence encoding a second promoter, wherein the second promoter drives expression of the sequence encoding the second guide RNA.

### V. THERAPEUTICALLY-EFFECTIVE RATIOS

In some embodiments, a first vector and a second vector are administered to a patient in a therapeutically effective ratio. As used herein, the term “ratio” may refer to a ratio of the amount of vector in a composition (concentration), amount delivered to a patient (dosage), amount available to a therapeutic site (bioavailability), amount expressed by a target cell (copy number), amount of modifications made (efficacy), amount of DNA, or number of coding sequences (e.g., sequences encoding a gRNA or a Cas9).

In some embodiments, the ratio of the first vector and the second vector is between 1:1 and 1:30. In other embodiments, the ratio of the first vector and the second vector is between 30:1 and 1:1. In some embodiments, the ratio of the first vector to the second vector is greater than 10:1. For example, the ratio of the first vector to the second vector may be about 11:1, about 12:1, about 13:1, about 14:1, about 15:1, about 16:1, about 17:1, about 18:1, about 19:1, about 20:1, about 25:1, about 30:1, about 35:1, about 40:1, about 50:1, about 75:1, or about 100:1. In some embodiments, the ratio of an AAV-gRNA vector to an AAV-Cas9 vector is greater than 10:1; for example, the ratio may be about 11:1, about 12:1, about 13:1, about 14:1, about 15:1, about 16:1, about 17:1, about 18:1, about 19:1, about 20:1, about 25:1, about 30:1, about 35:1, about 40:1, about 50:1, about 75:1, or about 100:1.

In some embodiments, between 5×10⁶ viral genomes (vg)/kilogram (kg) and 1×10¹⁵ vg/kg, inclusive of the endpoints, of the first and/or the second vector are administered to the patient. In some embodiments, between 5×10⁶ viral genomes (vg)/kilogram (kg) and 1×10¹⁰ vg/kg, inclusive of the endpoints, of the first and/or the second vector are administered to the patient. In some embodiments, at least 8×10⁷ viral genomes (vg)/kilogram (kg), at least 8×10⁸ viral genomes (vg)/kilogram (kg), at least 8×10⁹ viral genomes (vg)/kilogram (kg), or between 8×10⁷ viral genomes (vg)/kilogram (kg) and 8×10⁹ viral genomes (vg)/kilogram (kg) of the first and/or the second vector are administered to the patient.

In some embodiments, the Cas9 or Cpf1 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding Cas9 or Cpf1 and a guide RNA that targets ATXN2 are provided to a cell ex vivo before the cell is introduced or reintroduced to a patient.

In some embodiments, a composition comprises (i) a first nucleic acid sequence comprising a sequence encoding a first guide RNA targeting a first genomic target sequence, a sequence encoding a second guide RNA targeting a second genomic target sequence, a sequence encoding a first promoter wherein the first promoter drives expression of the sequence encoding the first guide RNA, and a sequence encoding a second promoter wherein the first promoter drives expression of the sequence encoding the second guide RNA, wherein the first genomic target sequence and the second genomic target sequence each comprise an ATXN2 sequence and (ii) a second nucleic acid sequence comprising a sequence encoding a promoter and a sequence encoding a Cas9 or a nuclease domain thereof. In some embodiments, the composition further comprises a pharmaceutically acceptable carrier.

In some embodiments, a composition comprises (i) a first vector comprising a nucleic acid sequence comprising a sequence encoding a first guide RNA targeting a first genomic target sequence, a sequence encoding a second guide RNA targeting a second genomic target sequence, a sequence encoding a first promoter wherein the first promoter drives expression of the sequence encoding the first guide RNA, and a sequence encoding a second promoter wherein the first promoter drives expression of the sequence encoding the second guide RNA, wherein the first genomic target sequence and the second genomic target sequence each comprise an ATXN2 sequence and (ii) a second vector comprising a nucleic acid sequence comprising a sequence encoding a promoter and a sequence encoding a Cas9 or a nuclease domain thereof. In some embodiments, at least one of the first vector and the second vectors are AAVs. In some embodiments, the composition further comprises a pharmaceutically acceptable carrier.

### VI. THERAPEUTIC METHODS AND USES

Also provided is a method for reducing ATXN2 expression, the method comprising contacting a cell with one or more compositions of the disclosure under conditions suitable for expression of at least a first guide RNA, and the Cas9 protein or a nuclease domain thereof, wherein at the first guide RNA forms a complex with the Cas9 protein or the nuclease domain thereof to form at least one guide RNA-Cas9 complex, wherein the at least one guide RNA-Cas9 complex reduces ATXN2 expression.

Also provided is a method of treating spinocerebellar ataxia type 2 in a subject in need thereof comprising administering to the subject a therapeutically effective amount of one or more compositions of the disclosure. In some embodiments, the composition is administered locally. In some embodiments, the composition is administered systemically. In some embodiments, the composition is administered by an intravenous infusion or injection. In some embodiments, the subject presents a sign or symptom of spinocerebellar ataxia type 2, which is reduced by the treatment. In some embodiments, the subject presents a neurological sign or symptom of spinocerebellar ataxia type 2 which is reduced by the treatment. In some embodiments, administration of the composition occurs prior to the subject presenting one or more progressive, later or neurological signs or symptoms of spinocerebellar ataxia type 2. In some embodiments, the subject is less than 10 years old, less than 5 years old, or less than 2 years old.

Also provided is the use of a therapeutically effective amount of one or more compositions of the disclosure for treating spinocerebellar ataxia type 2 in a subject in need thereof.

### VII. DELIVERY VECTORS

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

In some embodiments, a single viral vector is used to deliver a nucleic acid encoding a Cas9 or a Cpf1 and at least one gRNA to a cell. In some embodiments, Cas9 or Cpf1 is provided to a cell using a first viral vector and at least one gRNA is provided to the cell using a second viral vector.

In some embodiments, a single viral vector is used to deliver a nucleic acid encoding Cas9 or Cpf1 and at least one gRNA to a cell. In some embodiments, Cas9 or Cpf1 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (AS OR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### VIII. SPINOCEREBELLAR ATAXIA TYPE 2 (ATXN2)

Spinocerebellar ataxias (SCAs), also known as spinocerebellar atrophy or spinocerebellar degeneration, are progressive, degenerative, genetic diseases, each of which could be considered a neurological condition in its own right. An estimated 150,000 people in the United States have a diagnosis of spinocerebellar ataxia at any given time. SCA is hereditary, progressive, degenerative, and often fatal. There is no known effective treatment or cure. SCA can affect anyone of any age. The disease is caused by either a recessive or dominant gene. In many cases people are not aware that they carry a relevant gene until they have children who begin to show signs of having the disorder.

Spinocerebellar ataxia (SCA) is one of a group of genetic disorders characterized by slowly progressive incoordination of gait and is often associated with poor coordination of hands, speech, and eye movements. A review of different clinical features among SCA subtypes was recently published describing the frequency of non-cerebellar features, like parkinsonism, chorea, pyramidalism, cognitive impairment, peripheral neuropathy, seizures, among others. As with other forms of ataxia, SCA frequently results in atrophy of the cerebellum, loss of fine coordination of muscle movements leading to unsteady and clumsy motion, and other symptoms. The symptoms of an ataxia vary with the specific type and with the individual patient. In many cases a person with ataxia retains full mental capacity but progressively loses physical control.

The hereditary ataxias are categorized by mode of inheritance and causative gene or chromosomal locus. The hereditary ataxias can be inherited in an autosomal dominant, autosomal recessive, or X-linked manner Many types of autosomal dominant cerebellar ataxias for which specific genetic information is available are now known. Synonyms for autosomal-dominant cerebellar ataxias (ADCA) used prior to the current understanding of the molecular genetics were Marie's ataxia, inherited olivopontocerebellar atrophy, cerebello-olivary atrophy, or the more generic term “spinocerebellar degeneration.” (Spinocerebellar degeneration is a rare inherited neurological disorder of the central nervous system characterized by the slow degeneration of certain areas of the brain. There are three forms of spinocerebellar degeneration: Types 1, 2, 3. Symptoms begin during adulthood.)

Spinocerebellar ataxia 2 (SCA2) is a progressive disorder that causes symptoms including uncoordinated movement (ataxia), speech and swallowing difficulties, muscle wasting, slow eye movement, and sometimes dementia. Signs and symptoms usually begin in mid-adulthood but can appear any time from childhood to late-adulthood. SCA2 is caused by mutations in the ATXN2 gene and is inherited in an autosomal dominant manner Representative sequences are shown below.

| Transcript Variant: 1 | |
|---|---|
| (SEQ ID NO: 22) | |
| MRSAAAAPRSPAVATESRRFAAARWPGWRSLQRPARRSGRGGGG | |
| AAPGPYPSAAPPPPGPGPPPSRQSSPPSASDCFGSNGNGGGAFRPGSRRLLGLGGPPR | |
| PFVVLLLPLASPGAPPAAPTRASPLGARASPPRSGVSLARPAPGCPRPACEPVYGPLT | |
| MSLKPQQQQQQQQQQQQQQQQQQQQQQQPPPAAANVRKPGGSGLLASPAAAPSPSSSS | |
| VSSSSATAPSSVVAATSGGGRPGLGRGRNSNKGLPQSTISFDGIYANMRMVHILTSVV | |
| GSKCEVQVKNGGIYEGVFKTYSPKCDLVLDAAHEKSTESSSGPKREEIMESILFKCSD | |
| FVVVQFKDMDSSYAKRDAFTDSAISAKVNGEHKEKDLEPWDAGELTANEELEALENDV | |
| SNGWDPNDMFRYNEENYGVVSTYDSSLSSYTVPLERDNSEEFLKREARANQLAEEIES | |
| SAQYKARVALENDDRSEEEKYTAVQRNSSEREGHSINTRENKYIPPGQRNREVISWGS | |
| GRQNSPRMGQPGSGSMPSRSTSHTSDFNPNSGSDQRVVNGGVPWPSPCPSPSSRPPSR | |
| YQSGPNSLPPRAATPTRPPSRPPSRPSRPPSHPSAHGSPAPVSTMPKRMSSEGPPRMS | |
| PKAQRHPRNHRVSAGRGSISSGLEFVSHNPPSEAATPPVARTSPSGGTWSSVVSGVPR | |
| LSPKTHRPRSPRQNSIGNTPSGPVLASPQAGIIPTEAVAMPIPAASPTPASPASNRAV | |
| TPSSEAKDSRLQDQRQNSPAGNKENIKPNETSPSFSKAENKGISPVVSEHRKQIDDLK | |
| KFKNDFRLQPSSTSESMDQLLNKNREGEKSRDLIKDKIEPSAKDSFIENSSSNCTSGS | |
| SKPNSPSISPSILSNTEHKRGPEVTSQGVQTSSPACKQEKDDKEEKKDAAEQVRKSTL | |
| NPNAKEFNPRSFSQPKPSTTPTSPRPQAQPSPSMVGHQQPTPVYTQPVCFAPNMMYPV | |
| PVSPGVQPLYPIPMTPMPVNQAKTYRAVPNMPQQRQDQHHQSAMMHPASAAGPPIAAT | |
| PPAYSTQYVAYSPQQFPNQPLVQHVPHYQSQHPHVYSPVIQGNARMMAPPTHAQPGLV | |
| SSSATQYGAHEQTHAMYACPKLPYNKETSPSFYFAISTGSLAQQYAHPNATLHPHTPH | |
| PQPSATPTGQQQSQHGGSHPAPSPVQHHQHQAAQALHLASPQQQSAIYHAGLAPTPPS | |
| MTPASNTQSPQNSFPAAQQTVFTIHPSHVQPAYTNPPHMAHVPQAHVQSGMVPSHPTA | |
| HAPMMLMTTQPPGGPQAALAQSALQPIPVSTTAHFPYMTHPSVQAHHQQQL | |
| (SEQ ID NO: 23) | |
| 1 | acccccgaga aagcaaccca gcgcgccgcc cgctcctcac gtgtccctcc cggccccggg |
| 61 | gccacctcac gttctgcttc cgtctgaccc ctccgacttc cggtaaagag tccctatccg |
| 121 | cacctccgct cccacccggc gcctcggcgc gcccgccctc cgatgcgctc agcggccgca |
| 181 | gctcctcgga gtcccgcggt ggccaccgag tctcgccgct tcgccgcagc caggtggccc |
| 241 | gggtggcgct cgctccagcg gccggcgcgg cggagcgggc ggggcggcgg tggcgcggcc |
| 301 | ccgggaccgt atccctccgc cgcccctccc ccgcccggcc ccggcccccc tccctcccgg |
| 361 | cagagctcgc ctccctccgc ctcagactgt tttggtagca acggcaacgg cggcggcgcg |
| 421 | tttcggcccg gctcccggcg gctccttggt ctcggcgggc ctccccgccc cttcgtcgtc |
| 481 | ctccttctcc ccctcgccag cccgggcgcc cctccggccg cgccaacccg cgcctccccg |
| 541 | ctcggcgccc gcgcgtcccc gccgcgttcc ggcgtctcct tggcgcgccc ggctcccggc |
| 601 | tgtccccgcc cggcgtgcga gccggtgtat gggcccctca ccatgtcgct gaagccccag |
| 661 | cagcagcagc agcagcagca gcagcagcag cagcagcaac agcagcagca gcagcagcag |
| 721 | cagcagccgc cgcccgcggc tgccaatgtc cgcaagcccg gcggcagcgg ccttctagcg |
| 781 | tcgcccgccg ccgcgccttc gccgtcctcg tcctcggtct cctcgtcctc ggccacggct |
| 841 | ccctcctcgg tggtcgcggc gacctccggc ggcgggaggc ccggcctggg cagaggtcga |
| 901 | aacagtaaca aaggactgcc tcagtctacg atttcttttg atggaatcta tgcaaatatg |
| 961 | aggatggttc atatacttac atcagttgtt ggctccaaat gtgaagtaca agtgaaaaat |
| 1021 | ggaggtatat atgaaggagt ttttaaaact tacagtccga agtgtgattt ggtacttgat |
| 1081 | gccgcacatg agaaaagtac agaatccagt tcggggccga aacgtgaaga aataatggag |
| 1141 | agtattttgt tcaaatgttc agactttgtt gtggtacagt ttaaagatat ggactccagt |
| 1201 | tatgcaaaaa gagatgcttt tactgactct gctatcagtg ctaaagtgaa tggcgaacac |
| 1261 | aaagagaagg acctggagcc ctgggatgca ggtgaactca cagccaatga ggaacttgag |
| 1321 | gctttggaaa atgacgtatc taatggatgg gatcccaatg atatgtttcg atataatgaa |
| 1381 | gaaaattatg gtgtagtgtc tacgtatgat agcagtttat cttcgtatac agtgccctta |
| 1441 | gaaagagata actcagaaga atttttaaaa cgggaagcaa gggcaaacca gttagcagaa |
| 1501 | gaaattgagt caagtgccca gtacaaagct cgagtggccc tggaaaatga tgataggagt |
| 1561 | gaggaagaaa aatacacagc agttcagaga aattccagtg aacgtgaggg gcacagcata |
| 1621 | aacactaggg aaaataaata tattcctcct ggacaaagaa atagagaagt catatcctgg |
| 1681 | ggaagtggga gacagaattc accgcgtatg ggccagcctg gatcgggctc catgccatca |
| 1741 | agatccactt ctcacacttc agatttcaac ccgaattctg gttcagacca aagagtagtt |
| 1801 | aatggaggtg ttccctggcc atcgccttgc ccatctcctt cctctcgccc accttctcgc |
| 1861 | taccagtcag gtcccaactc tcttccacct cgggcagcca cccctacacg gccgccctcc |
| 1921 | aggcccccct cgcggccatc cagacccccg tctcacccct ctgctcatgg ttctccagct |
| 1981 | cctgtctcta ctatgcctaa acgcatgtct tcagaagggc ctccaaggat gtccccaaag |
| 2041 | gcccagcgac atcctcgaaa tcacagagtt tctgctggga ggggttccat atccagtggc |
| 2101 | ctagaatttg tatcccacaa cccacccagt gaagcagcta ctcctccagt agcaaggacc |
| 2161 | agtccctcgg ggggaacgtg gtcatcagtg gtcagtgggg ttccaagatt atcccctaaa |
| 2221 | actcatagac ccaggtctcc cagacagaac agtattggaa atacccccag tgggccagtt |
| 2281 | cttgcttctc cccaagctgg tattattcca actgaagctg ttgccatgcc tattccagct |
| 2341 | gcatctccta cgcctgctag tcctgcatcg aacagagctg ttaccccttc tagtgaggct |
| 2401 | aaagattcca ggcttcaaga tcagaggcag aactctcctg cagggaataa agaaaatatt |
| 2461 | aaacccaatg aaacatcacc tagcttctca aaagctgaaa acaaaggtat atcaccagtt |
| 2521 | gtttctgaac atagaaaaca gattgatgat ttaaagaaat ttaagaatga ttttaggtta |
| 2581 | cagccaagtt ctacttctga atctatggat caactactaa acaaaaatag agagggagaa |
| 2641 | aaatcaagag atttgatcaa agacaaaatt gaaccaagtg ctaaggattc tttcattgaa |
| 2701 | aatagcagca gcaactgtac cagtggcagc agcaagccga atagccccag catttcccct |
| 2761 | tcaatactta gtaacacgga gcacaagagg ggacctgagg tcacttccca aggggttcag |
| 2821 | acttccagcc cagcatgtaa acaagagaaa gacgataagg aagagaagaa agacgcagct |
| 2881 | gagcaagtta ggaaatcaac attgaatccc aatgcaaagg agttcaaccc acgttccttc |
| 2941 | tctcagccaa agccttctac taccccaact tcacctcggc ctcaagcaca acctagccca |
| 3001 | tctatggtgg gtcatcaaca gccaactcca gtttatactc agcctgtttg ttttgcacca |
| 3061 | aatatgatgt atccagtccc agtgagccca ggcgtgcaac ctttataccc aatacctatg |
| 3121 | acgcccatgc cagtgaatca agccaagaca tatagagcag taccaaatat gccccaacag |
| 3181 | cggcaagacc agcatcatca gagtgccatg atgcacccag cgtcagcagc gggcccaccg |
| 3241 | attgcagcca ccccaccagc ttactccacg caatatgttg cctacagtcc tcagcagttc |
| 3301 | ccaaatcagc cccttgttca gcatgtgcca cattatcagt ctcagcatcc tcatgtctat |
| 3361 | agtcctgtaa tacagggtaa tgctagaatg atggcaccac caacacacgc ccagcctggt |
| 3421 | ttagtatctt cttcagcaac tcagtacggg gctcatgagc agacgcatgc gatgtatgca |
| 3481 | tgtcccaaat taccatacaa caaggagaca agcccttctt tctactttgc catttccacg |
| 3541 | ggctcccttg ctcagcagta tgcgcaccct aacgctaccc tgcacccaca tactccacac |
| 3601 | cctcagcctt cagctacccc cactggacag cagcaaagcc aacatggtgg aagtcatcct |
| 3661 | gcacccagtc ctgttcagca ccatcagcac caggccgccc aggctctcca tctggccagt |
| 3721 | ccacagcagc agtcagccat ttaccacgcg gggcttgcgc caactccacc ctccatgaca |
| 3781 | cctgcctcca acacgcagtc gccacagaat agtttcccag cagcacaaca gactgtcttt |
| 3841 | acgatccatc cttctcacgt tcagccggcg tataccaacc caccccacat ggcccacgta |
| 3901 | cctcaggctc atgtacagtc aggaatggtt ccttctcatc caactgccca tgcgccaatg |
| 3961 | atgctaatga cgacacagcc acccggcggt ccccaggccg ccctcgctca aagtgcacta |
| 4021 | cagcccattc cagtctcgac aacagcgcat ttcccctata tgacgcaccc ttcagtacaa |
| 4081 | gcccaccacc aacagcagtt gtaaggctgc cctggaggaa ccgaaaggcc aaattccctc |
| 4141 | ctcccttcta ctgcttctac caactggaag cacagaaaac tagaatttca tttattttgt |
| 4201 | ttttaaaata tatatgttga tttcttgtaa catccaatag gaatgctaac agttcacttg |
| 4261 | cagtggaaga tacttggacc gagtagaggc atttaggaac ttgggggcta ttccataatt |
| 4321 | ccatatgctg tttcagagtc ccgcaggtac cccagctctg cttgccgaaa ctggaagtta |
| 4381 | tttatttttt aataaccctt gaaagtcatg aacacatcag ctagcaaaag aagtaacaag |
| 4441 | agtgattctt gctgctatta ctgctaaaaa aaaaaaaaaa aaaaaatcaa gacttggaac |
| 4501 | gcccttttac taaacttgac aaagtttcag taaattctta ccgtcaaact gacggattat |
| 4561 | tatttataaa tcaagtttga tgaggtgatc actgtctaca gtggttcaac ttttaagtta |
| 4621 | agggaaaaac ttttactttg tagataatat aaaataaaaa cttaaaaaaa atttaaaaaa |
| 4681 | taaaaaaagt tttaaaaact gaaaaaaaaa aa |
| Transcript Variant: 2 | |
| (SEQ ID NO: 24) | |
| MRMVHILTSVVGSKCEVQVKNGGIYEGVFKTYSPKCDLVLDAAH | |
| EKSTESSSGPKREEIMESILFKCSDFVVVQFKDMDSSYAKRDAFTDSAISAKVNGEHK | |
| EKDLEPWDAGELTANEELEALENDVSNGWDPNDMFRYNEENYGVVSTYDSSLSSYTVP | |
| LERDNSEEFLKREARANQLAEEIESSAQYKARVALENDDRSEEEKYTAVQRNSSEREG | |
| HSINTRENKYIPPGQRNREVISWGSGRQNSPRMGQPGSGSMPSRSTSHTSDFNPNSGS | |
| DQRVVNGGVPWPSPCPSPSSRPPSRYQSGPNSLPPRAATPTRPPSRPPSRPSRPPSHP | |
| SAHGSPAPVSTMPKRMSSEGPPRMSPKAQRHPRNHRVSAGRGSISSGLEFVSHNPPSE | |
| AATPPVARTSPSGGTWSSVVSGVPRLSPKTHRPRSPRQNSIGNTPSGPVLASPQAGII | |
| PTEAVAMPIPAASPTPASPASNRAVTPSSEAKDSRLQDQRQNSPAGNKENIKPNETSP | |
| SFSKAENKGISPVVSEHRKQIDDLKKFKNDFRLQPSSTSESMDQLLNKNREGEKSRDL | |
| IKDKIEPSAKDSFIENSSSNCTSGSSKPNSPSISPSILSNTEHKRGPEVTSQGVQTSS | |
| PACKQEKDDKEEKKDAAEQVRKSTLNPNAKEFNPRSFSQPKPSTTPTSPRPQAQPSPS | |
| MVGHQQPTPVYTQPVCFAPNMMYPVPVSPGVQPLYPIPMTPMPVNQAKTYRAVPNMPQ | |
| QRQDQHHQSAMMHPASAAGPPIAATPPAYSTQYVAYSPQQFPNQPLVQHVPHYQSQHP | |
| HVYSPVIQGNARMMAPPTHAQPGLVSSSATQYGAHEQTHAMYACPKLPYNKETSPSFY | |
| FAISTGSLAQQYAHPNATLHPHTPHPQPSATPTGQQQSQHGGSHPAPSPVQHHQHQAA | |
| QALHLASPQQQSAIYHAGLAPTPPSMTPASNTQSPQNSFPAAQQTVFTIHPSHVQPAY | |
| TNPPHMAHVPQCASEALARCGLEMRLSWIYLSEGYLAHVQSGMVPSHPTAHAPMMLMT | |
| TQPPGGPQAALAQSALQPIPVSTTAHFPYMTHPSVQAHHQQQL | |
| (SEQ ID NO: 25) | |
| 1 | cccgagaaag caacccagcg cgccgcccgc tcctcacgtg tccctcccgg ccccggggcc |
| 61 | acctcacgtt ctgcttccgt ctgacccctc cgacttccga ggtcgaaaca gtaacaaagg |
| 121 | actgcctcag tctacgattt cttttgatgg aatctatgca aatatgagga tggttcatat |
| 181 | acttacatca gttgttggct ccaaatgtga agtacaagtg aaaaatggag gtatatatga |
| 241 | aggagttttt aaaacttaca gtccgaagtg tgatttggta cttgatgccg cacatgagaa |
| 301 | aagtacagaa tccagttcgg ggccgaaacg tgaagaaata atggagagta ttttgttcaa |
| 361 | atgttcagac tttgttgtgg tacagtttaa agatatggac tccagttatg caaaaagaga |
| 421 | tgcttttact gactctgcta tcagtgctaa agtgaatggc gaacacaaag agaaggacct |
| 481 | ggagccctgg gatgcaggtg aactcacagc caatgaggaa cttgaggctt tggaaaatga |
| 541 | cgtatctaat ggatgggatc ccaatgatat gtttcgatat aatgaagaaa attatggtgt |
| 601 | agtgtctacg tatgatagca gtttatcttc gtatacagtg cccttagaaa gagataactc |
| 661 | agaagaattt ttaaaacggg aagcaagggc aaaccagtta gcagaagaaa ttgagtcaag |
| 721 | tgcccagtac aaagctcgag tggccctgga aaatgatgat aggagtgagg aagaaaaata |
| 781 | cacagcagtt cagagaaatt ccagtgaacg tgaggggcac agcataaaca ctagggaaaa |
| 841 | taaatatatt cctcctggac aaagaaatag agaagtcata tcctggggaa gtgggagaca |
| 901 | gaattcaccg cgtatgggcc agcctggatc gggctccatg ccatcaagat ccacttctca |
| 961 | cacttcagat ttcaacccga attctggttc agaccaaaga gtagttaatg gaggtgttcc |
| 1021 | ctggccatcg ccttgcccat ctccttcctc tcgcccacct tctcgctacc agtcaggtcc |
| 1081 | caactctctt ccacctcggg cagccacccc tacacggccg ccctccaggc ccccctcgcg |
| 1141 | gccatccaga cccccgtctc acccctctgc tcatggttct ccagctcctg tctctactat |
| 1201 | gcctaaacgc atgtcttcag aagggcctcc aaggatgtcc ccaaaggccc agcgacatcc |
| 1261 | tcgaaatcac agagtttctg ctgggagggg ttccatatcc agtggcctag aatttgtatc |
| 1321 | ccacaaccca cccagtgaag cagctactcc tccagtagca aggaccagtc cctcgggggg |
| 1381 | aacgtggtca tcagtggtca gtggggttcc aagattatcc cctaaaactc atagacccag |
| 1441 | gtctcccaga cagaacagta ttggaaatac ccccagtggg ccagttcttg cttctcccca |
| 1501 | agctggtatt attccaactg aagctgttgc catgcctatt ccagctgcat ctcctacgcc |
| 1561 | tgctagtcct gcatcgaaca gagctgttac cccttctagt gaggctaaag attccaggct |
| 1621 | tcaagatcag aggcagaact ctcctgcagg gaataaagaa aatattaaac ccaatgaaac |
| 1681 | atcacctagc ttctcaaaag ctgaaaacaa aggtatatca ccagttgttt ctgaacatag |
| 1741 | aaaacagatt gatgatttaa agaaatttaa gaatgatttt aggttacagc caagttctac |
| 1801 | ttctgaatct atggatcaac tactaaacaa aaatagagag ggagaaaaat caagagattt |
| 1861 | gatcaaagac aaaattgaac caagtgctaa ggattctttc attgaaaata gcagcagcaa |
| 1921 | ctgtaccagt ggcagcagca agccgaatag ccccagcatt tccccttcaa tacttagtaa |
| 1981 | cacggagcac aagaggggac ctgaggtcac ttcccaaggg gttcagactt ccagcccagc |
| 2041 | atgtaaacaa gagaaagacg ataaggaaga gaagaaagac gcagctgagc aagttaggaa |
| 2101 | atcaacattg aatcccaatg caaaggagtt caacccacgt tccttctctc agccaaagcc |
| 2161 | ttctactacc ccaacttcac ctcggcctca agcacaacct agcccatcta tggtgggtca |
| 2221 | tcaacagcca actccagttt atactcagcc tgtttgtttt gcaccaaata tgatgtatcc |
| 2281 | agtcccagtg agcccaggcg tgcaaccttt atacccaata cctatgacgc ccatgccagt |
| 2341 | gaatcaagcc aagacatata gagcagtacc aaatatgccc caacagcggc aagaccagca |
| 2401 | tcatcagagt gccatgatgc acccagcgtc agcagcgggc ccaccgattg cagccacccc |
| 2461 | accagcttac tccacgcaat atgttgccta cagtcctcag cagttcccaa atcagcccct |
| 2521 | tgttcagcat gtgccacatt atcagtctca gcatcctcat gtctatagtc ctgtaataca |
| 2581 | gggtaatgct agaatgatgg caccaccaac acacgcccag cctggtttag tatcttcttc |
| 2641 | agcaactcag tacggggctc atgagcagac gcatgcgatg tatgcatgtc ccaaattacc |
| 2701 | atacaacaag gagacaagcc cttctttcta ctttgccatt tccacgggct cccttgctca |
| 2761 | gcagtatgcg caccctaacg ctaccctgca cccacatact ccacaccctc agccttcagc |
| 2821 | tacccccact ggacagcagc aaagccaaca tggtggaagt catcctgcac ccagtcctgt |
| 2881 | tcagcaccat cagcaccagg ccgcccaggc tctccatctg gccagtccac agcagcagtc |
| 2941 | agccatttac cacgcggggc ttgcgccaac tccaccctcc atgacacctg cctccaacac |
| 3001 | gcagtcgcca cagaatagtt tcccagcagc acaacagact gtctttacga tccatccttc |
| 3061 | tcacgttcag ccggcgtata ccaacccacc ccacatggcc cacgtacctc agtgcgccag |
| 3121 | tgaggctctg gcaaggtgtg ggctagagat gcgactcagt tggatctatc tctcagaagg |
| 3181 | ctaccttgct catgtacagt caggaatggt tccttctcat ccaactgccc atgcgccaat |
| 3241 | gatgctaatg acgacacagc cacccggcgg tccccaggcc gccctcgctc aaagtgcact |
| 3301 | acagcccatt ccagtctcga caacagcgca tttcccctat atgacgcacc cttcagtaca |
| 3361 | agcccaccac caacagcagt tgtaaggctg ccctggagga accgaaaggc caaattccct |
| 3421 | cctcccttct actgcttcta ccaactggaa gcacagaaaa ctagaatttc atttattttg |
| 3481 | tttttaaaat atatatgttg atttcttgta acatccaata ggaatgctaa cagttcactt |
| 3541 | gcagtggaag atacttggac cgagtagagg catttaggaa cttgggggct attccataat |
| 3601 | tccatatgct gtttcagagt cccgcaggta ccccagctct gcttgccgaa actggaagtt |
| 3661 | atttattttt taataaccct tgaaagtcat gaacacatca gctagcaaaa gaagtaacaa |
| 3721 | gagtgattct tgctgctatt actgctaaaa aaaaaaaaaa aaaaaaatca agacttggaa |
| 3781 | cgccctttta ctaaacttga caaagtttca gtaaattctt accgtcaaac tgacggatta |
| 3841 | ttatttataa atcaagtttg atgaggtgat cactgtctac agtggttcaa cttttaagtt |
| 3901 | aagggaaaaa cttttacttt gtagataata taaaataaaa acttaaaaaa aatttaaaaa |
| 3961 | ataaaaaaag ttttaaaaac tgaaaaaaaa aaa |
| Transcript Variant: 3 | |
| (SEQ ID NO: 26) | |
| MRMVHILTSVVCDLVLDAAHEKSTESSSGPKREEIMESILFKCS | |
| DFVVVQFKDMDSSYAKRDAFTDSAISAKVNGEHKEKDLEPWDAGELTANEELEALEND | |
| VSNGWDPNDMFRYNEENYGVVSTYDSSLSSYTVPLERDNSEEFLKREARANQLAEEIE | |
| SSAQYKARVALENDDRSEEEKYTAVQRNSSEREGHSINTRENKYIPPGQRNREVISWG | |
| SGRQNSPRMGQPGSGSMPSRSTSHTSDFNPNSGSDQRVVNGGVPWPSPCPSPSSRPPS | |
| RYQSGPNSLPPRAATPTRPPSRPPSRPSRPPSHPSAHGSPAPVSTMPKRMSSEGPPRM | |
| SPKAQRHPRNHRVSAGRGSISSGLEFVSHNPPSEAATPPVARTSPSGGTWSSVVSGVP | |
| RLSPKTHRPRSPRQNSIGNTPSGPVLASPQAGIIPTEAVAMPIPAASPTPASPASNRA | |
| VTPSSEAKDSRLQDQRQNSPAGNKENIKPNETSPSFSKAENKGISPVVSEHRKQIDDL | |
| KKFKNDFRLQPSSTSESMDQLLNKNREGEKSRDLIKDKIEPSAKDSFIENSSSNCTSG | |
| SSKPNSPSISPSILSNTEHKRGPEVTSQGVQTSSPACKQEKDDKEEKKDAAEQVRKST | |
| LNPNAKEFNPRSFSQPKPSTTPTSPRPQAQPSPSMVGHQQPTPVYTQPVCFAPNMMYP | |
| VPVSPGVQPLYPIPMTPMPVNQAKTYRAVPNMPQQRQDQHHQSAMMHPASAAGPPIAA | |
| TPPAYSTQYVAYSPQQFPNQPLVQHVPHYQSQHPHVYSPVIQGNARMMAPPTHAQPGL | |
| VSSSATQYGAHEQTHAMYVSTGSLAQQYAHPNATLHPHTPHPQPSATPTGQQQSQHGG | |
| SHPAPSPVQHHQHQAAQALHLASPQQQSAIYHAGLAPTPPSMTPASNTQSPQNSFPAA | |
| QQTVFTIHPSHVQPAYTNPPHMAHVPQAHVQSGMVPSHPTAHAPMMLMTTQPPGGPQA | |
| ALAQSALQPIPVSTTAHFPYMTHPSVQAHHQQQL | |
| (SEQ ID NO: 27) | |
| 1 | cccgagaaag caacccagcg cgccgcccgc tcctcacgtg tccctcccgg ccccggggcc |
| 61 | acctcacgtt ctgcttccgt ctgacccctc cgacttccga tttcttttga tggaatctat |
| 121 | gcaaatatga ggatggttca tatacttaca tcagttgttt gtgatttggt acttgatgcc |
| 181 | gcacatgaga aaagtacaga atccagttcg gggccgaaac gtgaagaaat aatggagagt |
| 241 | attttgttca aatgttcaga ctttgttgtg gtacagttta aagatatgga ctccagttat |
| 301 | gcaaaaagag atgcttttac tgactctgct atcagtgcta aagtgaatgg cgaacacaaa |
| 361 | gagaaggacc tggagccctg ggatgcaggt gaactcacag ccaatgagga acttgaggct |
| 421 | ttggaaaatg acgtatctaa tggatgggat cccaatgata tgtttcgata taatgaagaa |
| 481 | aattatggtg tagtgtctac gtatgatagc agtttatctt cgtatacagt gcccttagaa |
| 541 | agagataact cagaagaatt tttaaaacgg gaagcaaggg caaaccagtt agcagaagaa |
| 601 | attgagtcaa gtgcccagta caaagctcga gtggccctgg aaaatgatga taggagtgag |
| 661 | gaagaaaaat acacagcagt tcagagaaat tccagtgaac gtgaggggca cagcataaac |
| 721 | actagggaaa ataaatatat tcctcctgga caaagaaata gagaagtcat atcctgggga |
| 781 | agtgggagac agaattcacc gcgtatgggc cagcctggat cgggctccat gccatcaaga |
| 841 | tccacttctc acacttcaga tttcaacccg aattctggtt cagaccaaag agtagttaat |
| 901 | ggaggtgttc cctggccatc gccttgccca tctccttcct ctcgcccacc ttctcgctac |
| 961 | cagtcaggtc ccaactctct tccacctcgg gcagccaccc ctacacggcc gccctccagg |
| 1021 | cccccctcgc ggccatccag acccccgtct cacccctctg ctcatggttc tccagctcct |
| 1081 | gtctctacta tgcctaaacg catgtcttca gaagggcctc caaggatgtc cccaaaggcc |
| 1141 | cagcgacatc ctcgaaatca cagagtttct gctgggaggg gttccatatc cagtggccta |
| 1201 | gaatttgtat cccacaaccc acccagtgaa gcagctactc ctccagtagc aaggaccagt |
| 1261 | ccctcggggg gaacgtggtc atcagtggtc agtggggttc caagattatc ccctaaaact |
| 1321 | catagaccca ggtctcccag acagaacagt attggaaata cccccagtgg gccagttctt |
| 1381 | gcttctcccc aagctggtat tattccaact gaagctgttg ccatgcctat tccagctgca |
| 1441 | tctcctacgc ctgctagtcc tgcatcgaac agagctgtta ccccttctag tgaggctaaa |
| 1501 | gattccaggc ttcaagatca gaggcagaac tctcctgcag ggaataaaga aaatattaaa |
| 1561 | cccaatgaaa catcacctag cttctcaaaa gctgaaaaca aaggtatatc accagttgtt |
| 1621 | tctgaacata gaaaacagat tgatgattta aagaaattta agaatgattt taggttacag |
| 1681 | ccaagttcta cttctgaatc tatggatcaa ctactaaaca aaaatagaga gggagaaaaa |
| 1741 | tcaagagatt tgatcaaaga caaaattgaa ccaagtgcta aggattcttt cattgaaaat |
| 1801 | agcagcagca actgtaccag tggcagcagc aagccgaata gccccagcat ttccccttca |
| 1861 | atacttagta acacggagca caagagggga cctgaggtca cttcccaagg ggttcagact |
| 1921 | tccagcccag catgtaaaca agagaaagac gataaggaag agaagaaaga cgcagctgag |
| 1981 | caagttagga aatcaacatt gaatcccaat gcaaaggagt tcaacccacg ttccttctct |
| 2041 | cagccaaagc cttctactac cccaacttca cctcggcctc aagcacaacc tagcccatct |
| 2101 | atggtgggtc atcaacagcc aactccagtt tatactcagc ctgtttgttt tgcaccaaat |
| 2161 | atgatgtatc cagtcccagt gagcccaggc gtgcaacctt tatacccaat acctatgacg |
| 2221 | cccatgccag tgaatcaagc caagacatat agagcagtac caaatatgcc ccaacagcgg |
| 2281 | caagaccagc atcatcagag tgccatgatg cacccagcgt cagcagcggg cccaccgatt |
| 2341 | gcagccaccc caccagctta ctccacgcaa tatgttgcct acagtcctca gcagttccca |
| 2401 | aatcagcccc ttgttcagca tgtgccacat tatcagtctc agcatcctca tgtctatagt |
| 2461 | cctgtaatac agggtaatgc tagaatgatg gcaccaccaa cacacgccca gcctggttta |
| 2521 | gtatcttctt cagcaactca gtacggggct catgagcaga cgcatgcgat gtatgtttcc |
| 2581 | acgggctccc ttgctcagca gtatgcgcac cctaacgcta ccctgcaccc acatactcca |
| 2641 | caccctcagc cttcagctac ccccactgga cagcagcaaa gccaacatgg tggaagtcat |
| 2701 | cctgcaccca gtcctgttca gcaccatcag caccaggccg cccaggctct ccatctggcc |
| 2761 | agtccacagc agcagtcagc catttaccac gcggggcttg cgccaactcc accctccatg |
| 2821 | acacctgcct ccaacacgca gtcgccacag aatagtttcc cagcagcaca acagactgtc |
| 2881 | tttacgatcc atccttctca cgttcagccg gcgtatacca acccacccca catggcccac |
| 2941 | gtacctcagg ctcatgtaca gtcaggaatg gttccttctc atccaactgc ccatgcgcca |
| 3001 | atgatgctaa tgacgacaca gccacccggc ggtccccagg ccgccctcgc tcaaagtgca |
| 3061 | ctacagccca ttccagtctc gacaacagcg catttcccct atatgacgca cccttcagta |
| 3121 | caagcccacc accaacagca gttgtaaggc tgccctggag gaaccgaaag gccaaattcc |
| 3181 | ctcctccctt ctactgcttc taccaactgg aagcacagaa aactagaatt tcatttattt |
| 3241 | tgtttttaaa atatatatgt tgatttcttg taacatccaa taggaatgct aacagttcac |
| 3301 | ttgcagtgga agatacttgg accgagtaga ggcatttagg aacttggggg ctattccata |
| 3361 | attccatatg ctgtttcaga gtcccgcagg taccccagct ctgcttgccg aaactggaag |
| 3421 | ttatttattt tttaataacc cttgaaagtc atgaacacat cagctagcaa aagaagtaac |
| 3481 | aagagtgatt cttgctgcta ttactgctaa aaaaaaaaaa aaaaaaaaat caagacttgg |
| 3541 | aacgcccttt tactaaactt gacaaagttt cagtaaattc ttaccgtcaa actgacggat |
| 3601 | tattatttat aaatcaagtt tgatgaggtg atcactgtct acagtggttc aacttttaag |
| 3661 | ttaagggaaa aacttttact ttgtagataa tataaaataa aaacttaaaa aaaatttaaa |
| 3721 | aaataaaaaa agttttaaaa actgaaaaaa aaaaa |

Early symptoms of spinocerebellar ataxia may include uncoordinated movement (ataxia) and leg cramps. Other symptoms may include tremor; decreased muscle tone; poor tendon reflexes; abnormal eye movements; dementia; dystonia and/or chorea; muscle twitches; nerve irritation and swelling (polyneuropathy); leg weakness; difficulty swallowing; bladder dysfunction; and parkinsonism.

Spinocerebellar ataxia 2 (SCA2) is inherited in an autosomal dominant manner This means that having one changed (mutated) copy of ATXN2 in each cell is enough to cause signs and symptoms of the condition. The ATXN2 gene mutations that cause SCA2 involve a DNA sequence called a ‘CAG trinucleotide repeat.’ It is made up of a series of three DNA building blocks (CAG stands for cytosine, adenine, and guanine) that appear multiple times in a row. The CAG sequence is normally repeated about 22 times in the gene, but it can be repeated up to 31 times without causing health problems. SCA2 develops in people who have 32 or more CAG repeats in the ATXN2 gene.

In most cases, an affected person inherits the mutated gene (with too many repeats) from an affected parent. However, in some cases, an affected person does not have an affected parent. People with an increased number of CAG repeats who don't develop SCA2 are still at risk of having children who will develop the disorder. This is because as the gene is passed down from parent to child, the number of CAG repeats often increases. In general, the more repeats a person has, the earlier symptoms begin. This phenomenon is called anticipation. People with 32 or 33 repeats tend to develop symptoms in late adulthood, while people with more than 45 repeats often have symptoms by their teens. For some reason, the number of repeats tend to increase more when the gene is inherited from a person's father than when inherited from a person's mother. Each child of an affected person has a 50% chance of inheriting the CAG repeat expansion.

Molecular genetic testing (analysis of DNA) is needed for a diagnosis of spinocerebellar ataxia 2 (SCA2). This testing detects abnormal CAG trinucleotide repeat expansions in the ATXN2 gene. Affected people (or people who will later develop symptoms of SCA2) have a copy of the ATXN2 gene that has 33 or more CAG repeats. This testing detects nearly 100% of cases of SCA2.

There is no cure for spinocerebellar ataxias, which are currently considered to be progressive and irreversible diseases, although not all types cause equally severe disability. In general, treatments are directed towards alleviating symptoms, not the disease itself. Many patients with hereditary or idiopathic forms of ataxia have other symptoms in addition to ataxia. Medications or other therapies might be appropriate for some of these symptoms, which could include tremor, stiffness, depression, spasticity, and sleep disorders, among others. Both onset of initial symptoms and duration of disease are variable. If the disease is caused by a polyglutamine trinucleotide repeat CAG expansion, a longer expansion may lead to an earlier onset and a more radical progression of clinical symptoms. Typically, a person afflicted with this disease will eventually be unable to perform daily tasks (ADLs). However, rehabilitation therapists can help patients to maximize their ability of self-care and delay deterioration to certain extent. Researchers are exploring multiple avenues for a cure including RNAi and the use of Stem Cells and several other avenues.

Physical therapists can assist patients in maintaining their level of independence through therapeutic exercise programmes One recent research report demonstrated a gain of 2 SARA points (Scale for the Assessment and Rating of Ataxia) from physical therapy. In general, physical therapy emphasises postural balance and gait training for ataxia patients. General conditioning such as range-of-motion exercises and muscle strengthening would also be included in therapeutic exercise programmes Research showed that spinocerebellar ataxia 2 (SCA2) patients with a mild stage of the disease gained significant improvement in static balance and neurological indices after six months of a physical therapy exercise training program. Occupational therapists may assist patients with incoordination or ataxia issues through the use of adaptive devices. Such devices may include a cane, crutches, walker, or wheelchair for those with impaired gait. Other devices are available to assist with writing, feeding, and self care if hand and arm coordination are impaired. A randomised clinical trial revealed that an intensive rehabilitation program with physical and occupational therapies for patients with degenerative cerebellar diseases can significantly improve functional gains in ataxia, gait, and activities of daily living. Some level of improvement was shown to be maintained 24 weeks post-treatment. Speech language pathologists may use both behavioral intervention strategies as well as augmentative and alternative communication devices to help patients with impaired speech.

In particular, treatment of spinocerebellar ataxia 2 (SCA2) is supportive and aims to help the affected person maintain their independence and avoid injury. It is recommended that people with SCA2 remain physically active, maintain a healthy weight, use adaptive equipment as needed, and avoid alcohol and medications that affect cerebellar function. Adaptive equipment may include canes or other devices to help with walking and mobility. People with SCA2 may develop difficulty speaking and may need to use computerized devices or writing pads to help with communication. Levodopa may be prescribed to help with some of the movement problems (e.g., rigidity and tremor), and magnesium may improve muscle cramping.

### IX. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

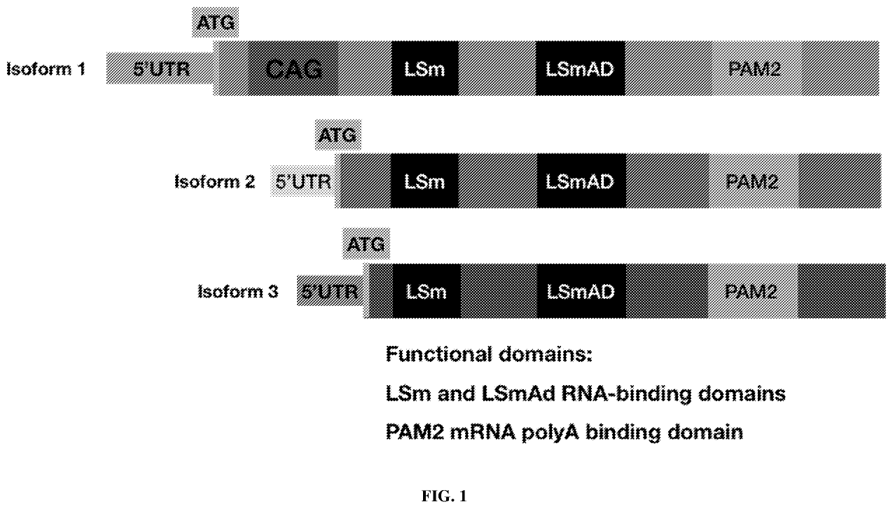

FIG. 1 provides a diagram of the ATXN2 protein isoform variant 1, isoform variant 2, and isoform variant 3 targeted by the CRISPR/Cas9 system. All isoform variants contain functional domains: LSm, LSmAd, and PAM2. The mRNA expression of these three isoform variants were targeted with gRNAs and SpCas9. A subset of gRNAs were more specific for targeting isoform variant 1, which harbors the CAG trinucleotide repeat expansion.

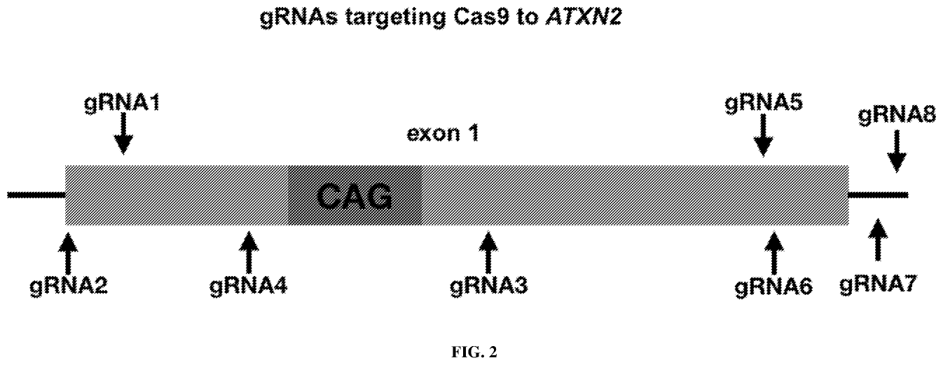

FIG. 2 provides a diagram of the CRISPR/Cas9 system's gRNA locations targeting SpCas9 to exon 1 of ATXN2 with the CAG trinucleotide repeat. gRNA3, gRNA5, and gRNA6 are used as single gRNAs. gRNA1, gRNA2, gRNA4, gRNA7, and gRNA8 are used as dual co-expressed gRNAs in combinations that can also include gRNA3, gRNA5, and gRNA6. For example, co-expression of gRNA2 and gRNA3 excise the CAG repeat and/or reduce ATXN2 protein levels. gRNA3 expressed alone result in indels and reduces ATXN2 protein levels. These gRNAs were tested to reduce human ATXN2 protein levels in HEK 293 cells and BAC-ATXN2-Q72 SCA2 transgenic mice, which contains a 150 kB transgene of the full-length human ATXN2 gene with a 72 CAG trinucleotide repeat as well as endogenous regulatory sequence.

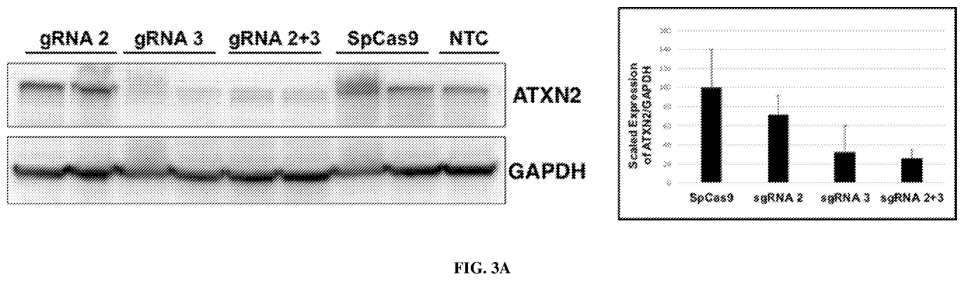

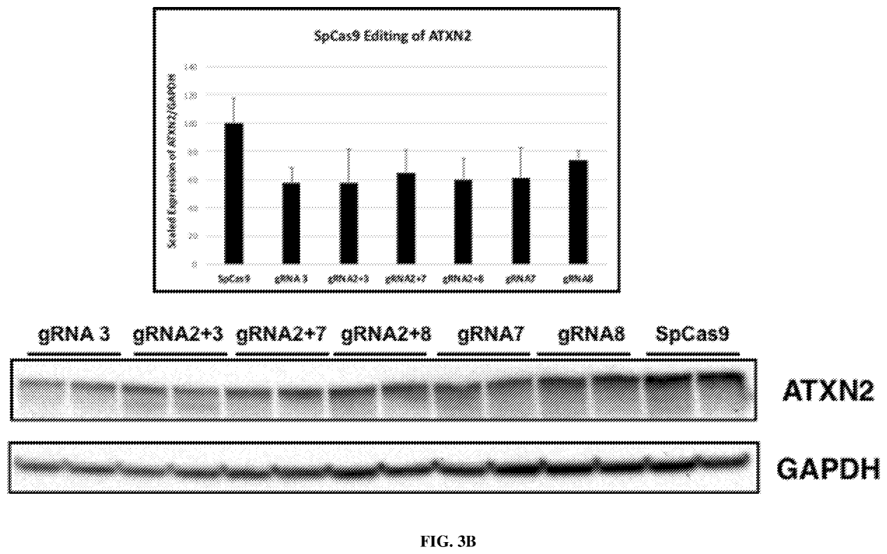

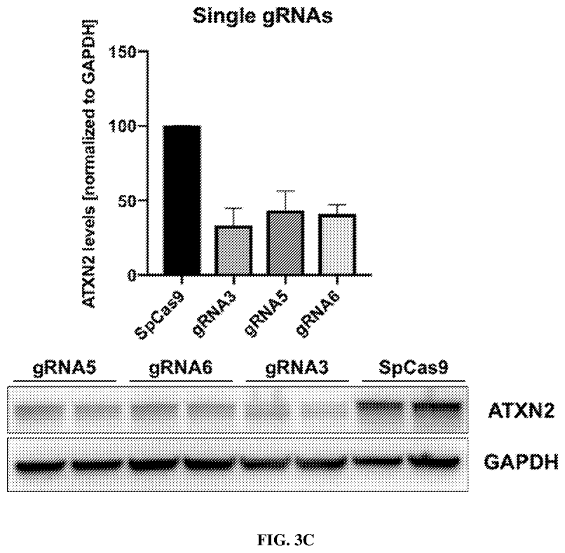

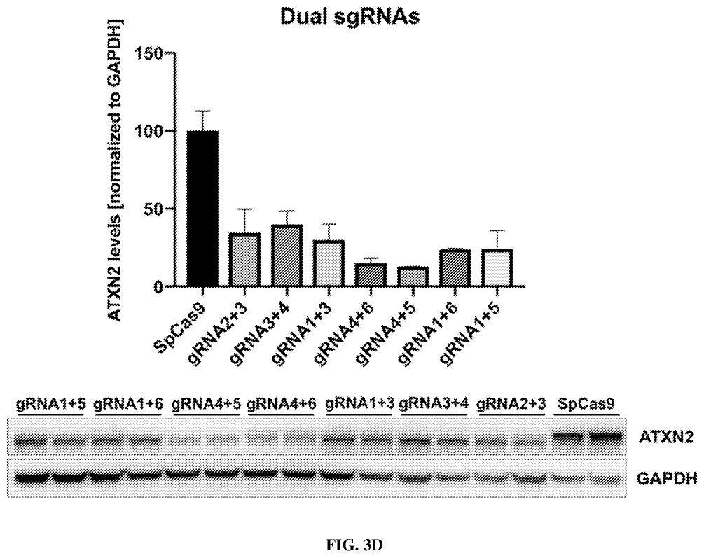

FIGS. 3A, 3B, 3C and 3D provide data showing single gRNAs with SpCas9 and dual gRNAs co-expressed with SpCas9 reduced ATXN2 protein expression levels in HEK 293 cells.

The plasmid pX330 containing the SpCas9 and gRNA expression cassettes was provided by Dr. Feng Zhang and used as a template for further modifications. To determine transfection efficacy and for selecting positive transfected cells, a CMV reporter cassette expressing eGFP/P2A/puromycin fusion protein was cloned downstream of the SpCas9 expression cassette. For all gRNAs, the guide complementary sequences were cloned using a single cloning step with a pair of partially complementary oligonucleotides. The oligo pairs encoding the genomic complementary guide sequences were annealed and ligated into the BbsI cloning site upstream and in frame with the invariant scaffold of the gRNA sequence. A human U6 Pol3 promoter was used to drive expression of the gRNA sequences.

Plasmids expressing gRNAs were transfected into HEK 293 cells (normal ATXN2 CAG trinucleotide repeat length) with SpCas9 and compared to a SpCas9 only transfection control. HEK 293 cells were cultured in DMEM with 10% FBS at 37 degrees Celsius. Lipofectamine 2000 was used to transfect 500 ng of plasmid DNA in 2×10⁵ cells per well in a 24-well plate and cultured for 24 hours. Transfected cells were positively selected by 3 mM puromycin (Life Technologies) treatment for 48 hours.

Following puromycin selection cells were cultured to confluency, washed once with cold PBS, and then lysed in RIPA buffer (50 mM Tris pH 8.0, 150 mM NaCl, 1% Triton X100, 0.1% SDS, 0.5% Na deoxycholate) with 1 complete protease inhibitor, followed by incubation on ice for 20 minutes with occasional vortexing. Cell lysate debris was removed by centrifuging in 1.5 mL Eppendorf tubes for 20 minutes at 20,000 g at 4 degrees

Celsius, and supernatants were stored at -80 degrees Celsius until used. Protein concentrations were quantified with Pierce BCA protein assay kit (ThermoFisher Scientific). SDS-PAGE was performed with 20 micrograms of protein in Laemmli sample buffer with DTT, denatured for 10 minutes at 95 degrees Celsius, and loaded on a 12% Bis-Tris polyacrylamide gel (Bio-Rad) with MES running buffer (Bio-Rad) and run at 150 volts, and transferred overnight onto polyvinylidene fluoride (PVDF) membranes at 4 degrees Celsius. Blots were blocked in 5% milk in 1×TBST for 2 hours at room temperature, primary antibodies mouse anti-ATXN2 (611378, BD Biosciences 1:500 dilution) or mouse anti-GAPDH (6C5 ab8245, Abcam 1:15,000 dilution) in 2% milk in 1×TBST for 1 hour at room temperature, and corresponding goat anti-mouse HRP-labeled secondary antibodies (Abcam, 1:15,000 dilution) in 2% milk in 1×TBST for 1 hour at room temperature. Blots were developed with Amersham ECL Prime detection reagents (GE Healthcare Life Sciences). Densitometry was done with ImageJ for quantifications of ATXN2 levels relative to GAPDH with ChemiDoc Imaging System (Bio-Rad) and Image Lab analysis software.

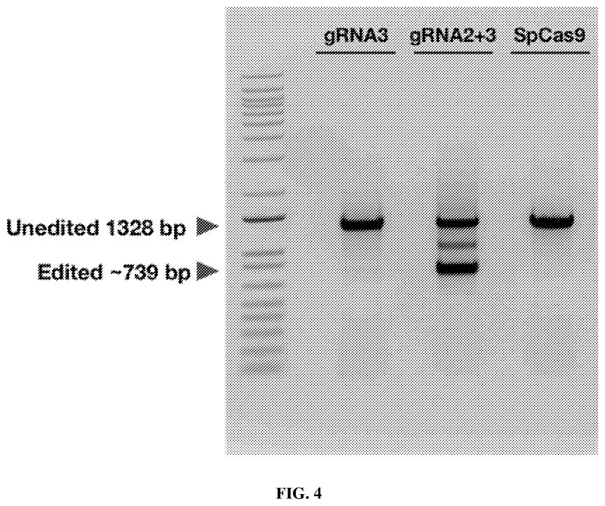

FIG. 4 provides data showing co-expression of gRNA2 and gRNA3 with SpCas9 resulted in a deletion band (the lower band below the unedited band) at the predicted size of 739 base pairs that corresponds in size to the sequence length between the targeted DNA in HEK 293 cells. As expected, gRNA3 alone did not result in a deletion band similar to the SpCas9 only control unedited band.

After plasmid transfection and puromycin selection, DNA was isolated using phenol-chloroform extraction followed by ethanol precipitation by established protocols. 150 nanograms of genomic DNA was PCR amplified using the following conditions: 500 nM forward primer 5′ GCT TCT CTC CTG TCC TCC ACA CCA G 3′ (SEQ ID NO: 41), 500 nM reverse primer 5′ CCA TCG CCG CTA CCC GAG AAC 3′ (SEQ ID NO: 42), 200 uM dNTPs, and 1 unit of BIOLASE Taq Polymerase (Bioline). PCR was performed with an initial 95 degree Celsius denaturation for 5 minutes followed by 35 cycles of 95 degree Celsius for 30 seconds, 58 degree Celsius for 30 seconds, and 72 degree Celsius for 1.5 minutes. PCR product was separated by size by gel electrophoresis (1% agarose) in TAE buffer at 100 volts for 1 hour. Samples were amplified and electrophoresed with a SpCas9 only transfection control that shows amplification of the unedited target.

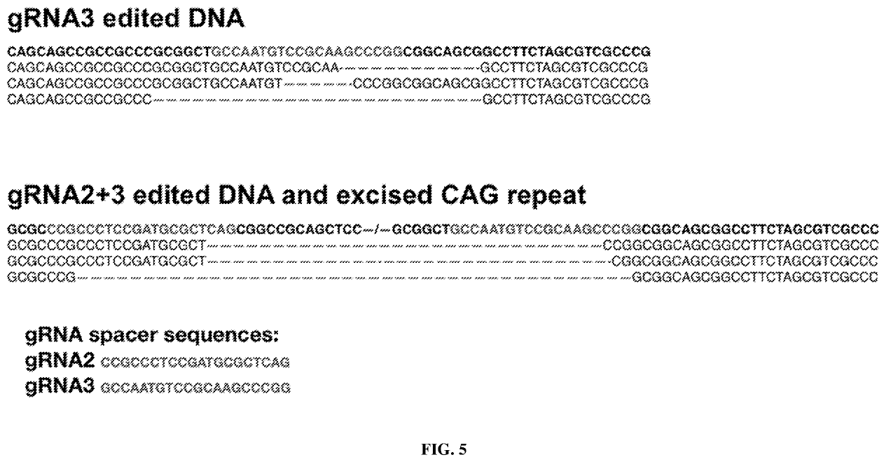

FIG. 5 provides data showing indels from 3 clones after editing with gRNA3 with SpCas9 and deletions from 3 clones after editing with co-expression of gRNA2 and gRNA3 with SpCas9 compared to the above unedited reference DNA sequence. gRNA3 resulted in small indels, and co-expression of gRNA2 and gRNA3 resulted in deletion of the CAG trinucleotide repeat sequence.

After plasmid transfection and puromycin selection in HEK 293 cells, isolated genomic DNA were PCR amplified (see FIG. 4 PCR conditions), separated by size by gel electrophoresis (0.8% agarose) in TAE buffer at 100 volts for 1 hour, unedited and deletion bands were gel purified, cloned into TOPO plasmids using the TOPO TA Cloning Kit and subsequently transformed into DH5alpha competent cells. Individual colonies were analyzed using Sanger sequencing.

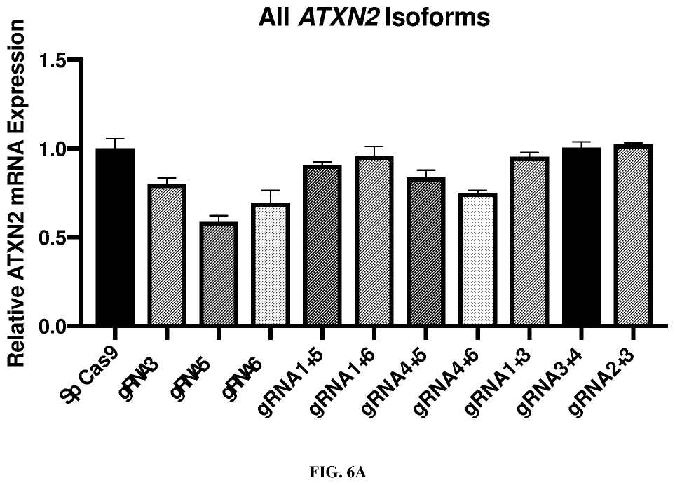

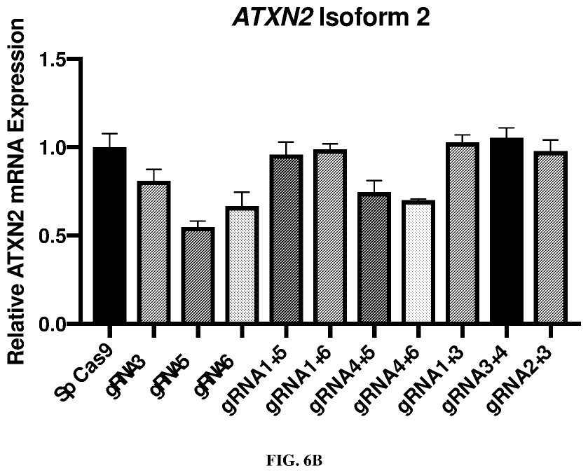

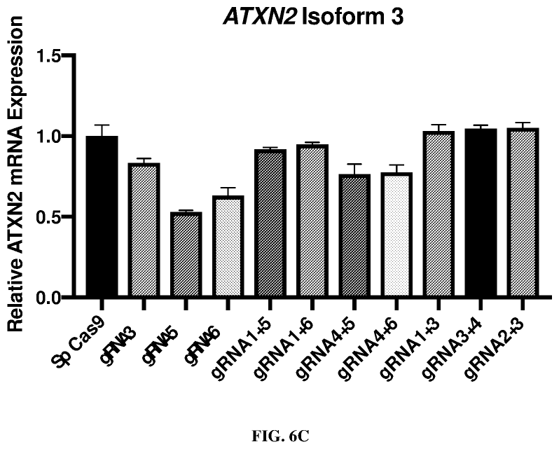

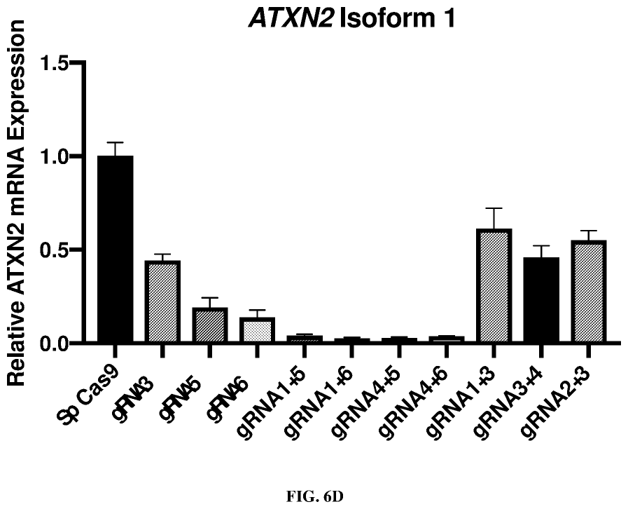

FIGS. 6A, 6B, 6C and 6D provide data showing single gRNAs with SpCas9 and dual co-expressed gRNAs with SpCas9 reduced ATXN2 mRNA expression levels compared to SpCas9 only control in HEK 293 cells. FIGS. 6A, 6B, 6C and 6D show single gRNA5 and gRNA6, and dual co-expression of gRNA4 and gRNA5, and gRNA4 and gRNA6, reduced mRNA levels of all ATXN2 isoforms, including isoform variant 1, isoform variant 2 and isoform variant 3. FIGS. 6A, 6B, 6C and 6D show single gRNA3 and dual co-expression of gRNA1 and gRNA5, gRNA1 and gRNA6, gRNA1 and gRNA3, gRNA3 and gRNA4, and gRNA2 and gRNA3, more specifically reduced mRNA levels of ATXN2 isoform variant 1.

After plasmid transfection and puromycin selection in HEK 293 cells, total RNA was extracted using TRIzol (Life Technologies) according to the manufacturer's protocol, with the exception of 1 uL Glycoblue (Life Technologies) in addition to the aqueous phase on the isopropanol precipitation step and a single wash with cold 70% ethanol. RNA samples were quantified by Nanodrop spectrophotometry. cDNAs were generated using 1 microgram of total RNA for RT-PCR. For measuring the expression of all ATXN2 isoforms, isoform variant 2, and isoform variant 3, random hexamers were used for RT-PCR. For measuring the expression of ATXN2 isoform variant 1, isoform variant 1 specific primers, forward 5′ TCG TCC TCG GTC TCC TCG TCC 3′ (SEQ ID NO: 43) and reverse 5′ ACT GGC ATG GGC GTC ATA GG 3′ (SEQ ID NO: 44), were used for RT-PCR. Quantitative PCR (qPCR) of the RT-PCR generated cDNA was performed for all ATXN2 isoforms and isoform variant 1 with primer/probe set Hs00268077_m1 (ATXN2 TaqMan gene expression assay catalog #4331182 Applied Biosystems ThermoFisher Scientific), isoform variant 2 with forward primer 5′ GCC CAC GTA CCT CAG TG 3′ (SEQ ID NO: 45) and reverse primer 5′ AGG TAG CCT TCT GAG AGA TAG A 3′ (SEQ ID NO: 46) and 6FAM-probe 5′ CAA GGT GTG GGC TAG AGA TGC GAC 3′ (SEQ ID NO: 47), isoform variant 3 with forward primer 5′ AGC CTG GTT TAG TAT CTT CTT CAG 3′ (SEQ ID NO: 48) and reverse primer 5′ GCG TTA GGG TGC GCA TA 3′ (SEQ ID NO: 49) and 6FAM-probe 5′ ATG CGA TGT ATG TTT CCA CGG GCT 3′ (SEQ ID NO: 50). GAPDH primer probe set Hs99999905_m1 (GAPDH TaqMan gene expression assay catalog #4331182 Applied Biosystems ThermoFisher Scientific) was used to normalize ATXN2 mRNA expression levels.

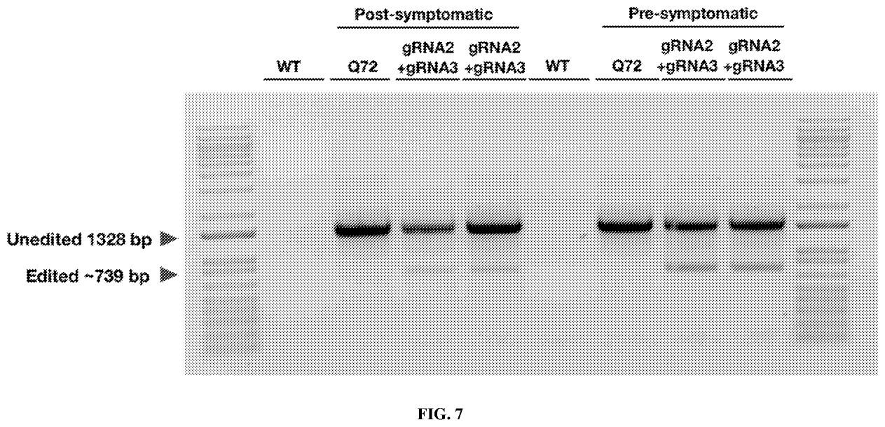

FIG. 7 provides data showing co-expression of gRNA2 and gRNA3 with SpCas9 in both pre-symptomatic mice (6 weeks old) and post-symptomatic mice (13 weeks old) resulted in a deletion band (the lower band below the unedited band) at the predicted size of 739 base pairs that corresponds in size to the sequence length between the targeted DNA in BAC-ATXN2-Q72 SCA2 mice. Wildtype littermates were used as a non-transgenic PCR control.

For in vivo studies (FIGS. 7-11), two different recombinant adeno-associated viral (rAAV) vectors were generated: one expressed SpCas9 and one expressed the gRNAs. SpCas9 was expressed under the control of the Mecp2 or Synl promoter and cloned upstream of a polyA sequence (px551 plasmid provided by Dr. Feng Zhang) The gRNA expression cassettes were moved into an AAV shuttle plasmid (G0619 pFBAAVmcsCMVeGFP SV40pA) containing an eGFP gene under the control of the CMV promoter and upstream of an SV40 polyA sequence. All rAAV plasmid shuttles have AAV2 inverted terminal repeat sequences. rAAV vectors were produced by standard linear polyethylenimine (PEI) max transfection method in HEK 293 cells by using the AdHelper, AAV1 Rep/Cap trans-packaging plasmid, and AAV genomic cargo shuttle plasmids. Vectors were purified on an iodixanol gradient by ultracentrifugation, titers were determined by qPCR using SpCas9 or eGFP SYBR green primers, and vector purity was tested by silver stain. HEK 293 cells were provided by the Children's Hospital of Philadelphia Research Vector Core.

All animal protocols were approved by the Children's Hospital of Philadelphia Animal Care Institutional and Use Committee. BAC-ATXN2-Q72 SCA2 transgenic mice were provided by Dr. Stefan Pulst and rederived by Jackson Laboratory. The BAC-ATXN2-Q72 line was maintained on the FVB background. Mice were genotyped using primers specific for the human BAC-ATXN2-Q72 transgene. Hemizygous and age-matched wildtype littermates were used for the experiments. Mice were housed in a controlled temperature environment on a 12-hour light/dark cycle. Food and water were provided ad libitum.

Mice were injected at 6 weeks of age (pre-symptomatic) or 13 weeks of age (post-symptomatic) with a combination of 1:1 rAAV2/1-Mecp2-SpCas9 and rAAV2/1- hU6gRNA-CMV-eGFP vectors, For rAAV injections, mice were anesthetized with isoflurane, and 8 uL of rAAV mixture (4uL/hemisphere) stereotaxically injected bilaterally to the deep cerebellar nuclei (DCN) at 0.2 mL/min (coordinates between −6.1 and -6.0 mm caudal to bregma,±2.0 mm from midline, and −2.2 mm deep from cerebellar surface). For DNA and protein analysis, after 4 weeks mice were anesthetized with a ketamine and xylazine mix and transcardially perfused with 20 mL of ice-cold PBS. Brains were removed and cut into 1-mm-thick sagittal slices, GFP positive cerebellar tissue was dissected for enrichment, flash frozen in liquid nitrogen and stored at -80 degrees Celsius until use. Frozen samples were weighed and lysed with 20 uL/mg of RIPA buffer for western blot analysis.

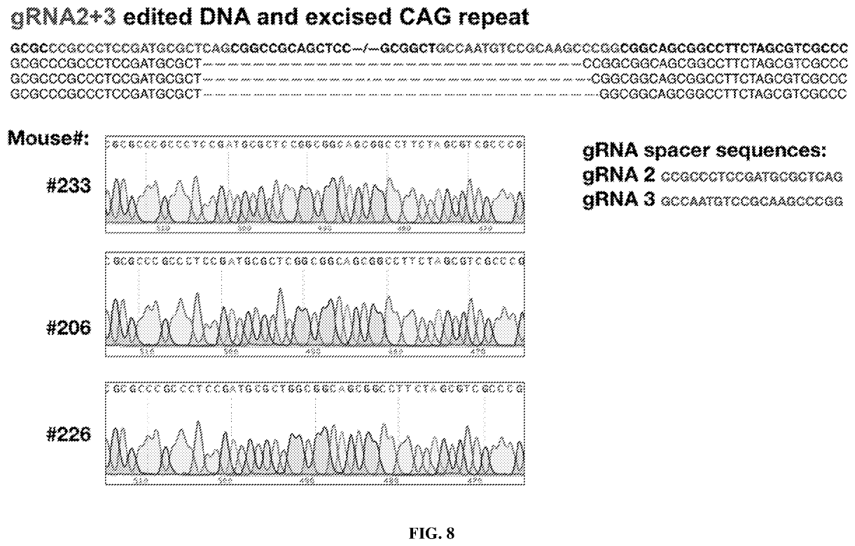

FIG. 8 provides data showing 3 clones after editing with co-expression of gRNA2 and gRNA3 with SpCas9 compared to the above unedited reference DNA sequence (Mouse IDs #233 and #226 were injected at 6 weeks old and ID #206 was injected at 13 weeks old). Co-expression of gRNA2 and gRNA3 with SpCas9 resulted in deletion of the CAG repeat sequence. Four weeks after mice were injected and brains were harvested, isolated genomic DNA were PCR amplified, TOPO cloned, and Sanger Sequenced (see FIG. 5 conditions).

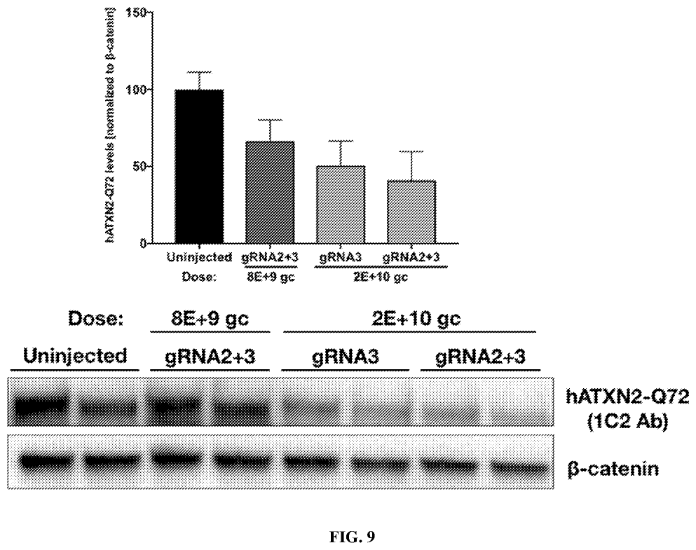

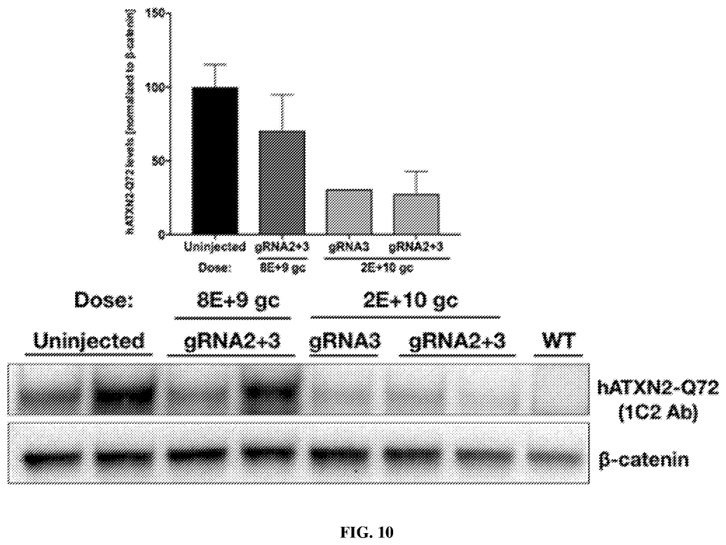

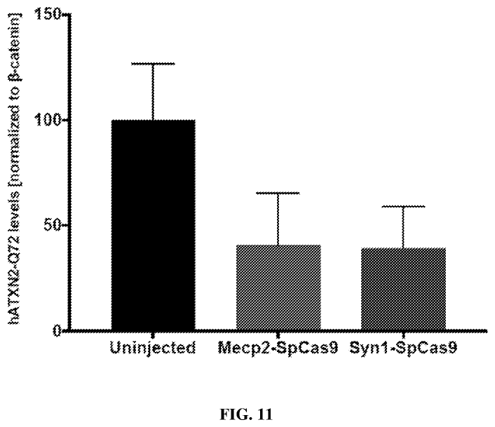

FIG. 9 and FIG. 10 provide data showing dose-dependent reduction of human ATXN2-Q72 protein levels after infusion of AAV1-SpCas9 and AAV1-gRNA vectors into the cerebellum of pre-onset and post-onset BAC-ATXN2-Q72 mice, respectively. Vector doses are shown as total genome copies infused bilaterally. FIG. 11 provides data showing a comparison between Mecp2 and Synl neuronal-specific promoters driving SpCas9 expression similarly reduced human ATXN2-Q72 protein levels after infusion of AAV1-SpCas9 and AAV1-gRNA3 into the cerebellum of 8 week old BAC-ATXN2-Q72 mice.

Protein lysates were loaded on 3-8% Tris-Acetate gels (Bio-Rad) and MOPS running buffer (Bio-Rad) (see FIG. 3 for western blot methods). Primary antibodies used were mouse anti-polyglutamine-expansion 5TF1-1C2 for hATXN2-Q72 (MAB1574, 1:3000 dilution, Millipore Sigma) diluted in 5% milk 1×TBST and rabbit anti-beta catenin (Ab2365, 1:5000 dilution, Abcam) in 2% milk 1×TBST for 2 hours at room temperature, and corresponding goat anti-mouse or anti-rabbit HRP-labeled secondary antibodies (Abcam, 1:15,000 dilution) in 5% milk or 2% milk 1×TBST for 1 hour at room temperature.

### Example 2—Results

FIGS. 3A, 3B, 3C and 3D provide examples of designed gRNAs targeting exon 1 of human ATXN2. The ability of the gRNAs to reduce human ATXN2 protein was tested in HEK 293 cells. gRNAs with SpCas9 were transfected into HEK 293 cells.

FIG. 4 and FIG. 5 provide examples of gRNAs editing ATXN2 DNA sequence. The ability of the gRNAs with SpCas9 to cut DNA and result in indels and deletions of the CAG repeat was tested in HEK 293 cells.

FIGS. 6A, 6B, 6C and 6D provide examples of gRNAs reducing ATXN2 mRNA expression levels. The ability of gRNAs with SpCas9 to reduce all ATXN2 isoform variants, including isoform variant 1, variant 2 and variant3 was tested in HEK 239 cells. The ability of gRNAs with SpCas9 to more specifically reduce ATXN2 isoform variant 1 was tested in HEK 293 cells.

FIG. 7 and FIG. 8 provide examples of gRNAs editing human ATXN2 DNA in vivo. The ability of the gRNAs with SpCas9 to edit DNA and result in deletion of the CAG repeat was tested in the cerebellum of BAC-ATXN2-Q72 SCA2 mice.

FIG. 9 and FIG. 10 provide examples of gRNAs reducing human ATXN2-Q72 protein levels in vivo. The ability of the gRNAs with SpCas9 to reduce human ATXN2-Q72 protein levels in a dose-dependent manner was tested in the cerebellum of BAC-ATXN2-Q72 SCA2 mice.

FIG. 11 provides an example of comparing two neuron-specific promoters driving SpCas9 with gRNA3 to reduce human ATXN2-Q72 protein levels. The ability of the two promoters to drive expression of SpCas9 and reduce human ATXN2-Q72 protein levels was tested in the cerebellum of BAC-ATXN2-Q72 SCA2 mice.

The results indicate that exon 1 of human ATXN2 with a CAG trinucleotide repeat is targeted by the CRISPR/Cas9 system. The gRNAs that have shown editing to date flank the CAG repeat sequence. Editing of this region with a fully active SpCas9 nuclease has been shown. Further, the ATXN2 trinucleotide CAG repeat was sequenced in a cell line and a mouse model that was edited using gRNAs. The sequences showed indels with single gRNAs and deletions of the CAG trinucleotide repeat with dual co-expressed gRNAs. The gRNAs reduced levels of human ATXN2 mRNA and ATXN2 protein levels in cells. A subset of gRNAs more specifically reduced mRNA levels of ATXN2 isoform variant 1. A subset of gRNAs reduced mRNA levels of all ATXN2 isoform variants, including isoform variant 1, isoform variant 2, and isoform variant 3. The gRNAs reduced levels of human ATXN2-Q72 protein levels in transgenic mice. A comparison between two neuron-specific promoters driving SpCas9 expression similarly reduced levels of human ATXN2-Q72 protein levels in transgenic mice.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Benvenisty and Neshif, Proc. Natl. Acad. Sci. USA, 83:9551-9555, 1986.
Hersdorffer et al., DNA Cell Biol., 9:713-723, 1990.
Markowitz et al., J. Virol., 62:1120-1124, 1988.
Roux et al., Proc. Natl. Acad. Sci. USA, 86:9079-9083, 1989.
Wang et al., Cell, 153:910-910, 2013.

What is claimed is:

1. A composition comprising:
a first vector comprising a nucleic acid comprising:
a sequence encoding at least a first guide RNA targeting a genomic target sequence according to SEQ ID NO: 1, 3, 4, 5, 6, 7 or 8;
a sequence encoding a first promoter, wherein the first promoter drives expression of the sequence encoding the first guide RNA, and
a second vector comprising a nucleic acid comprising:
a sequence encoding a Cas9 or a nuclease domain thereof;
a sequence encoding a second promoter, wherein the second promoter drives expression of the sequence encoding a Cas9 or a nuclease domain thereof.

2. The composition of claim 1, wherein a ratio of the first vector to the second vector in the composition is between 1:1 and 1:30.

3. The composition of claim 1, wherein the first vector comprises at least one sequence encoding a second guide RNA targeting a genomic target sequence according to SEQ ID NO:
2, or SEQ ID NO: 1, 3, 4, 5, 6, 7 or 8 that differs from the first guide RNA; and at least one sequence encoding a third promoter, wherein the third promoter drives expression of the at least one sequence encoding the second guide RNA.

4. The composition of claims 1-2, wherein the first vector and/or the second vector comprises a sequence isolated or derived from an adeno-associated virus (AAV), such as adeno-associated virus 1 (AAV1).

5. The composition of any one of claims 1-4, wherein the sequence encoding the first promoter comprises or consists of a U6 promoter.

6. The composition of any one of claims 3-5, wherein the sequence encoding the third promoter comprises or consists of a U6 promoter.

7. The composition of any one of claims 1-6, wherein the sequence encoding the second promoter comprises or consists of a Mecp2 promoter or a Synl promoter.

8. The composition of any one of claims 1-7, wherein the composition comprises between 5×10⁶ viral genomes (vg)/kilogram (kg) and 1×10¹⁵ vg/kg, inclusive of the endpoints, of the first vector.

9. The composition of any one of claims 1-7, wherein the composition comprises 1×10⁷ viral genomes (vg)/kilogram (kg) to 1×10¹⁰ viral genomes (vg)/kilogram (kg), inclusive of the endpoints, of the first vector.

10. The composition of any one of claims 1-9, further comprising a pharmaceutically acceptable carrier.

11. A composition comprising a first vector comprising a nucleic acid comprising:
a sequence encoding at least a first guide RNA targeting a genomic target sequence according to SEQ ID NO: 1, 3, 4, 5, 6, 7 or 8;
a sequence encoding a first promoter, wherein the first promoter drives expression of the sequence encoding the first guide RNA.

12. The composition of claim 11, wherein the first vector comprises at least one sequence encoding a second guide RNA targeting a genomic target sequence according to SEQ ID NO:
2, or SEQ ID NO: 1, 3, 4, 5, 6, 7 or 8 that differs from the first guide RNA; and at least one sequence encoding a second promoter, wherein the second promoter drives expression of the at least one sequence encoding the second guide RNA.

13. The composition of claims 11-12, wherein the first vector comprises a sequence isolated or derived from an adeno-associated virus (AAV).

14. The composition of claims 11-12, wherein the first vector comprises a sequence isolated or derived from an adeno-associated virus 1 (AAV1).

15. The composition of any one of claims 11-14, wherein the sequence encoding the first promoter comprises or consists of a U6 promoter.

16. The composition of any one of claims 13-15, wherein the sequence encoding the second promoter comprises or consists of a U6 promoter.

17. The composition of any one of claims 11-16, wherein the composition comprises between 5×10⁶ viral genomes (vg)/kilogram (kg) and 1×10¹⁵ vg/kg, inclusive of the endpoints, of the first vector.

18. The composition of any one of claims 11-16, wherein the composition comprises 1×10⁷ viral genomes (vg)/kilogram (kg) to 1×10¹⁰ viral genomes (vg)/kilogram (kg), inclusive of the endpoints, of the first vector.

19. The composition of any one of claims 11-18, further comprising a pharmaceutically acceptable carrier.

20. The composition of any one of claims 11-19, wherein said first vector further comprises a nucleic acid comprising:
a sequence encoding a Cas9 or a nuclease domain thereof;
a sequence encoding a third promoter, wherein the third promoter drives expression of the sequence encoding a Cas9 or a nuclease domain thereof.

21. A method of preventing, treating, slowing the onset of, or slowing the progression of spinocerebellar ataxia type 2 in a subject in need thereof comprising administering to the subject a therapeutically effective amount a first vector comprising a nucleic acid comprising:
a sequence encoding at least a first guide RNA targeting a genomic target sequence according to SEQ ID NO: 1, 3, 4, 5, 6, 7 or 8;
a sequence encoding a first promoter, wherein the first promoter drives expression of the sequence encoding the first guide RNA, and
a second vector comprising a nucleic acid comprising:
a sequence encoding a Cas9 or a nuclease domain thereof;
a sequence encoding a second promoter, wherein the second promoter drives expression of the sequence encoding a Cas9 or a nuclease domain thereof.

22. The method of claim 21, wherein the first vector comprises at least one sequence encoding a second guide RNA targeting a genomic target sequence according to SEQ ID NO:
2, or SEQ ID NO: 1, 3, 4, 5, 6, 7 or 8 that differs from the first guide RNA; and at least one sequence encoding a third promoter, wherein the third promoter drives expression of the at least one sequence encoding the second guide RNA.

23. The method of claim 21, wherein a ratio of the first vector to the second vector in the composition is between 1:1 and 1:30.

24. The method of claim 21, wherein the composition is administered locally, such as to brain tissue.

25. The method of claim 21, wherein the composition is administered systemically.

26. The method of claim 21, wherein the composition is administered by an intravenous infusion or injection.

27. The method of any one of claims 21-26, wherein the subject is a neonate, an infant, a child, a young adult, or an adult.

28. The method of any one of claims 21-27, wherein the subject has been diagnosed as having spinocerebellar ataxia type 2.

29. The method of any one of claims 21-27, wherein the subject is a genetic carrier for spinocerebellar ataxia type 2.

30. The method of any one of claims 21-29, wherein the subject is male.

31. The method of any one of claims 21-29, wherein the subject is female.

32. The method of any one of claims 21-31, wherein the subject is an adult.

33. The method of claim 32, wherein the adult is at least 18 years old.

34. The method of claim 32, wherein the adult is at least 25 years old.

35. The method of any one of claims 21-31, wherein the subject is a child.

36. The method of claims 35, wherein the child is less than 18 years of age.

37. The method of claims 35, wherein the child is 20 kg or less.

38. The method of any one of claims 21-29, wherein the subject is an infant.

39. The method of claim 38, wherein the subject is less than 2 years old.

40. The method of any one of claims 21-39, wherein upon administering the therapeutically effective amount of the composition, the subject produces a minimal immune response to the composition.

41. The method of claim 40, wherein the effective amount comprises between 5×10⁶ viral genomes (vg)/kilogram (kg) and 1×10¹⁵ vg/kg, inclusive of the endpoints, of the first vector.

42. The method of claim 40, wherein the composition comprises 1×10⁷ viral genomes (vg)/kilogram (kg) to 1×10¹⁰ viral genomes (vg)/kilogram (kg), inclusive of the endpoints, of the first vector.

43. The method of any one of claims 21-42, wherein the administration of the therapeutically effective amount of the composition is provided as a single dose or provided within a single medical procedure.

44. The method of any one of claims 21-42, wherein the administration of the therapeutically effective amount of the composition is provided as multiple doses or provided over multiple medical procedures.

45. The method of claims 21-44, wherein the administration results in the reduction of ATXN2 protein expression, but not ATXN2 mRNA.

46. The method of claim 45, wherein reduction of ATXN2 protein expression is by about 55%-70% as compared to pre-treatment levels in a targeted cell.

47. The method of claims 21-44, wherein the administration results in the introduction of indels near an ATXN2 target sequence.

48. The method of claims 21-44, wherein the administration results in the deletion of a CAG repeat in an ATXN2 target sequence.


SEQUENCE LISTING (50 sequences)

SEQ ID NO: 1 - DNA, 20 bp, Artificial sequence
  Synthetic oligo
gccaatgtccgcaagcccgg

SEQ ID NO: 2 - DNA, 20 bp, Artificial sequence
  Synthetic oligo
ccgccctccgatgcgctcag

SEQ ID NO: 3 - DNA, 20 bp, Artificial sequence
  Synthetic oligo
gcgagactcggtggccaccg

SEQ ID NO: 4 - DNA, 20 bp, Artificial sequence
  Synthetic oligo
accaaaacagtctgaggcgg

SEQ ID NO: 5 - DNA, 20 bp, Artificial sequence
  Synthetic oligo
gtcgccgcgaccaccgagga

SEQ ID NO: 6 - DNA, 20 bp, Artificial sequence
  Synthetic oligo
ggtcgccgcgaccaccgagg

SEQ ID NO: 7 - DNA, 20 bp, Artificial sequence
  Synthetic oligo
gtgacccgccgggctacccg

SEQ ID NO: 8 - DNA, 20 bp, Artificial sequence
  Synthetic oligo
gcccaccccgggtagcccgg

SEQ ID NO: 9 - protein, 1307 aa, Acidaminococcus
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 10 - protein, 1228 aa, Lachnospiraceae bacterium ND2006
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 11 - protein, 41 aa, Artificial sequence
  Syntheic peptide
RMRKFKNKGKDTAELRRRRVEVSVELRKAKKDEQILKRRNV

SEQ ID NO: 12 - protein, 8 aa, Artificial sequence
  Syntheic peptide
VSRKRPRP

SEQ ID NO: 13 - protein, 8 aa, Artificial sequence
  Syntheic peptide
PPKKARED

SEQ ID NO: 14 - protein, 8 aa, Artificial sequence
  Syntheic peptide
PQPKKKPL

SEQ ID NO: 15 - protein, 12 aa, Artificial sequence
  Syntheic peptide
SALIKKKKKMAP

SEQ ID NO: 16 - protein, 5 aa, Artificial sequence
  Syntheic peptide
DRLRR

SEQ ID NO: 17 - protein, 6 aa, Artificial sequence
  Syntheic peptide
KQKKRK

SEQ ID NO: 18 - protein, 10 aa, Artificial sequence
  Syntheic peptide
RKLKKKIKKL

SEQ ID NO: 19 - protein, 10 aa, Artificial sequence
  Syntheic peptide
REKKKFLKRR

SEQ ID NO: 20 - protein, 20 aa, Artificial sequence
  Syntheic peptide
KRKGDEVDGVDEVAKKKSKK

SEQ ID NO: 21 - protein, 17 aa, Artificial sequence
  Syntheic peptide
RKCLQAGMNLEARKTKK

SEQ ID NO: 22 - protein, 1313 aa, Homo sapiens
MRSAAAAPRSPAVATESRRFAAARWPGWRSLQRPARRSGRGGGGAAPGPYPSAAPPPPGPGPPPSRQSSPPSASDCFGSNGNGGGAFRPGSRRLLGLGGPPRPFVVLLLPLASPGAPPAAPTRASPLGARASPPRSGVSLARPAPGCPRPACEPVYGPLTMSLKPQQQQQQQQQQQQQQQQQQQQQQQPPPAAANVRKPGGSGLLASPAAAPSPSSSSVSSSSATAPSSVVAATSGGGRPGLGRGRNSNKGLPQSTISFDGIYANMRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 23 - DNA, 4712 bp, Homo sapiens
acccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 24 - protein, 1073 aa, Homo sapiens
MRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQCASEALARCGLEMRLSWIYLSEGYLAHVQSGMVPSHPTAHAPMMLMTTQPPGGPQAALAQSALQPIPVSTTAHFPYMTHPSVQAHHQQQL

SEQ ID NO: 25 - DNA, 3993 bp, Homo sapiens
cccgagaaagcaacccagcgcgccgcccgctcctcacgtgtccctcccggccccggggccacctcacgttctgcttccgtctgacccctccgacttccgaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcagtgcgccagtgaggctctggcaaggtgtgggctagagatgcgactcagttggatctatctctcagaaggctaccttgctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 26 - protein, 1006 aa, Homo sapiens
MRMVHILTSVVCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYVSTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 27 - DNA, 3755 bp, Homo sapiens
cccgagaaagcaacccagcgcgccgcccgctcctcacgtgtccctcccggccccggggccacctcacgttctgcttccgtctgacccctccgacttccgatttcttttgatggaatctatgcaaatatgaggatggttcatatacttacatcagttgtttgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgtttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 28 - DNA, 67 bp, Homo sapiens
cagcagccgccgcccgcggctgccaatgtccgcaagcccggcggcagcggccttctagcgtcgcccg

SEQ ID NO: 29 - DNA, 53 bp, Artificial Sequence
  Edited gene sequence
cagcagccgccgcccgcggctgccaatgtccgcaagccttctagcgtcgcccg

SEQ ID NO: 30 - DNA, 61 bp, Artificial Sequence
  Edited gene sequence
cagcagccgccgcccgcggctgccaatgtcccggcggcagcggccttctaggcgtcgcccg

SEQ ID NO: 31 - DNA, 33 bp, Artificial Sequence
  Edited gene sequence
cagcagccgccgcccgccttctagcgtcgcccg

SEQ ID NO: 32 - DNA, 88 bp, Homo sapiens
gcgcccgccctccgatgcgctcagcggccgcagctccgcggctgccaatgtccgcaagcccggcggcagcggccttctagcgtcgccc

SEQ ID NO: 33 - DNA, 50 bp, Artificial Sequence
  Edited gene sequence
gcgcccgccctccgatgcgctccggcggcagcggccttctagcgtcgccc

SEQ ID NO: 34 - DNA, 49 bp, Artificial Sequence
  Edited gene sequence
gcgcccgccctccgatgcgctcggcggcagcggccttctagcgtcgccc

SEQ ID NO: 35 - DNA, 33 bp, Artificial Sequence
  Edited gene sequence
gcgcccggcggcagcggccttctagcgtcgccc

SEQ ID NO: 36 - DNA, 49 bp, Artificial Sequence; identical to SEQ ID NO: 34
  Edited gene sequence

SEQ ID NO: 37 - DNA, 48 bp, Artificial Sequence
  Edited gene sequence
gcgcccgccctccgatgcgctggcggcagcggccttctagcgtcgccc

SEQ ID NO: 38 - DNA, 52 bp, Artificial Sequence
  Edited gene sequence
cgcgcccgccctccgatgcgctccggcggcagcggccttctagcgtcgcccg

SEQ ID NO: 39 - DNA, 51 bp, Artificial Sequence
  Edited gene sequence
cgcgcccgccctccgatgcgctcggcggcagcggccttctagcgtcgcccg

SEQ ID NO: 40 - DNA, 50 bp, Artificial Sequence
  Edited gene sequence
cgcgcccgccctccgatgcgctggcggcagcggccttctagcgtcgcccg

SEQ ID NO: 41 - DNA, 25 bp, Artificial Sequence
  Synthetic primer
gcttctctcctgtcctccacaccag

SEQ ID NO: 42 - DNA, 21 bp, Artificial Sequence
  Synthetic primer
ccatcgccgctacccgagaac

SEQ ID NO: 43 - DNA, 21 bp, Artificial Sequence
  Synthetic primer
tcgtcctcggtctcctcgtcc

SEQ ID NO: 44 - DNA, 20 bp, Artificial Sequence
  Synthetic primer
actggcatgggcgtcatagg

SEQ ID NO: 45 - DNA, 17 bp, Artificial Sequence
  Synthetic primer
gcccacgtacctcagtg

SEQ ID NO: 46 - DNA, 22 bp, Artificial Sequence
  Synthetic primer
aggtagccttctgagagataga

SEQ ID NO: 47 - DNA, 24 bp, Artificial Sequence
  Synthetic probe
caaggtgtgggctagagatgcgac

SEQ ID NO: 48 - DNA, 24 bp, Artificial Sequence
  Synthetic primer
agcctggtttagtatcttcttcag

SEQ ID NO: 49 - DNA, 17 bp, Artificial Sequence
  Synthetic primer
gcgttagggtgcgcata

SEQ ID NO: 50 - DNA, 24 bp, Artificial Sequence
  Synthetic probe
atgcgatgtatgtttccacgggct